(19) United States
(12) Patent Application Publication
(10) Pub. No.: US 2006/0211792 A1
(43) Pub. Date: Sep. 21, 2006

(54) Triangulenium fluorescent dyes and polymers comprising such dyes

(76) Inventor: Bo Wegge Laursen, (US)

Correspondence Address: BROWDY AND NEIMARK, P.L.L.C.;624 NINTH STREET, NW, SUITE 300, WASHINGTON, DC, 20001-5303, US

(21) Appl. No.: 10/566,757

(22) PCT Filed: Jul. 30, 2004
(86) PCT No.: PCT/DK04/00518

§ 371 Date: Feb. 1, 2006

(30) Foreign Application Priority Data
Aug. 1, 2003 (DK) PA 2003 01121
Aug. 1, 2003 (US) 60491527

(51) Int. Cl. C07D 491/14; C07D 493/14; C08K 5/34; C08K 5/48

(52) U.S. Cl. 524/90; 549/381; 546/38

(57) ABSTRACT

The invention relates to fluorescent dyes comprising a triangulene structure in the form of a ring system of six, six-membered rings fused in a triangular fashion. The dyes are useful for visualisation of chemical compounds including polymer matrices for e.g. chromatographic separation and isolation of desirable chemical moieties, and for chemical library synthesis.

All patent and non-patent references cited below are hereby incorporated herein. This application is a non-provisional of U.S. provisional application Ser. No. 60/491,529 filed on Aug. 1, 2004, which is hereby incorporated by reference in its entirety.

### TECHNICAL FIELD OF THE INVENTION

The present invention is in the field of fluorescent dyes comprising a triangulene structure in the form of a ring system of six, six-membered rings fused in a triangular fashion. The dyes are useful for visualisation of chemical compounds including polymer matrices for e.g. chromatographic separation and isolation of desirable chemical moieties, and for chemical library synthesis.

### BACKGROUND OF THE INVENTION

The term triangulene is the trivial name for the rather large and diverse class of compounds characterized by a ring system of six, six-membered rings fused in a triangular fashion. The triangulenes can be regarded as build up by fusion of six rings or as triphenyl methane bridged in all ortho positions. In systematic naming the ring system is a dibenzopyrene comprising a cationic moiety.

Only a few triangulenium salts have been reported in the literature. The compounds are reported to be stable carbenium ions. One example of a triangulenium compound is 4,8,12-trioxa-4,8,12,12c-tetrahydrodibenzo[cd,mn]pyrenium (B. W. Laursen, F. C. Krebs, M. F. Nielsen, K. Bechgaard, J. B. Christensen, N. Harrit, J. Am. Chem. Soc. 1998, 120 12255-12263; Laursen, B. W., University of Copenhagen/Risø National Laboratory, ISBN 87-550-2897-7, 2001; http://www.risoe.dk/rispubl/POUpolpdf/ris-r-1275.pdf).

Triangulenium dyes carrying dialkylamino groups in positions 2, 6, and 10 have recently been disclosed (Laursen, ibid). Such compounds are different from triangulenium compounds without electron donating substituents located in the para-position (i.e. positions 2, 6 and 10). The lowest electronic transition is characterised by a fully allowed charge transfer transition. The transition promotes a transfer of the electron density from the donating amino groups towards the cationic center.

The sharp, low-energy absorption bands in the visible region display extinction coefficients above 1×10⁵ M⁻¹ cm⁻¹ are well separated form UV transitions.

### SUMMARY OF THE INVENTION

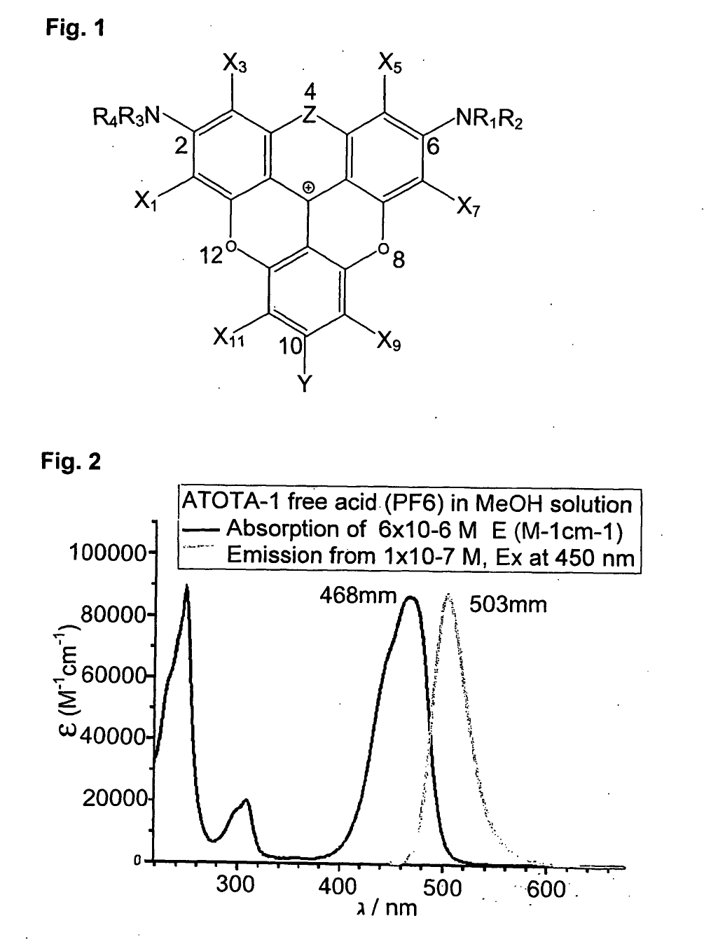

In one aspect of the invention, the dyes are substituted triangulenium dyes, described by the general structure in FIG. 1, in which at least one of the functional groups allows the dye to be covalently attached to other molecules or particles.

This type of dye display intense absorptions in visible region and fluoresces with high quantum efficiency.

The fluorescent dye compounds according to the invention are stable towards acid, base, reductive conditions, oxidative conditions. Accordingly, the dye compounds can be used as fluorophores (labels/tags) in assays operated under harsh chemical conditions.

The flexible synthesis schemes allow incorporation of one or more substituents optionally comprising a reactive group. At least one substituent comprising a reactive group is incorporated.

The term “reactive group” as used herein is a chemical group preferably selected from the group consisting of hydroxy, primary amine, secondary amine, carboxy, acid chloride, sulfonyl chloride, sulphonate, nitro, cyano, isothiocyanate, halogen, phosphonyl, sulphonyl, sulfamyl, carbonyl, and thiolyl, vinyl, acrylate, methacrylate, acrylamide, methyl oxethane, ethyl oxethane, and ethylene oxide.

The presence of two or more different reactive groups makes it possible for the dye compound to act simultaneously as a dye and as a linker capable of linking the dye compound to e.g. a beaded polymer or a biomolecule.

Definitions

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical.

Where the term “alkyl” is used, either alone or within other terms such as “haloalkyl” and “alkylsulfonyl”, it embraces linear or branched radicals having one to about twenty carbon atoms or, preferably, one to about twelve carbon atoms. Preferred alkyl radicals are “lower alkyl” radicals having one to about ten carbon atoms, such as lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like. Branched chain isomers of straight chain alkyl groups, include, but are not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)CH(CH₃)₂, —CH(CH₂CH₃)CH(CH₃)CH(CH₃)(CH₂CH₃), and others. When substituted, the “alkyl” or “lower alkyl” can comprise one or more radicals selected from the group of radicals consisting of hydroxy, primary amine, carboxy, acid chloride, sulfonyl chloride, sulphonate, nitro, cyano, isothiocyanate, halogen, phosphonyl, sulphonyl, sulfamyl, carbonyl, and thiolyl.

The term “alkenyl” embraces linear or branched radicals having at least one carbon-carbon double bond of two to about twenty carbon atoms, such as from two to about twelve carbon atoms, for example from two to about eight carbon atoms. Preferred alkyl radicals are “lower alkenyl” radicals having two to about six carbon atoms. Examples of such radicals include ethenyl, n-propenyl, butenyl, and the like. When substituted, the “alkenyl” or “lower alkenyl” can comprise one or more radicals selected from the group of radicals consisting of hydroxy, primary amine, carboxy, acid chloride, sulfonyl chloride, sulphonate, nitro, cyano, isothiocyanate, halogen, phosphonyl, sulphonyl, sulfamyl, carbonyl, and thiolyl.

The term “halo” means halogens such as fluorine, chlorine, bromine or iodine atoms. The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” preferably embraces radicals having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl. The “haloalkyl” or “lower haloalkyl” can optionally be further substituted. When further substituted, the “haloalkyl” or “lower haloalkyl” can further comprise one or more radicals selected from the group of radicals consisting of hydroxy, primary amine, carboxy, acid chloride, sulfonyl chloride, sulphonate, nitro, cyano, isothiocyanate, phosphonyl, sulphonyl, sulfamyl, carbonyl, and thiolyl.

The term “hydroxyalkyl” embraces linear or branched alkyl radicals having from one to about ten carbon atoms any one of which may be substituted with one or more hydroxyl radicals. Hydroxyalkyl radicals can be “lower hydroxyalkyl” radicals preferably having one to six carbon atoms and one or more hydroxyl radicals. Examples of such radicals include hydroxymethyl, hydroxyethyl, hydroxypropyl, hydroxybutyl and hydroxyhexyl. The “hydroxyalkyl” or “lower hydroxyalkyl” can optionally be further substituted. When further substituted, the “hydroxyalkyl” or “lower hydroxyalkyl” can further comprise one or more radicals selected from the group of radicals consisting of primary amine, carboxy, acid chloride, sulfonyl chloride, sulphonate, nitro, cyano, isothiocyanate, halogen, phosphonyl, sulphonyl, sulfamyl, carbonyl, and thiolyl.

The terms “alkoxy” and “alkoxyalkyl” embrace linear or branched oxy-containing radicals each having alkyl portions of one to about ten carbon atoms, such as methoxy radical. Alkoxy radicals can be “lower alkoxy” radicals having one to six carbon atoms. Examples of such radicals include methoxy, ethoxy, propoxy, butoxy and tert-butoxy. The term “alkoxyalkyl” also embraces alkyl radicals having two or more alkoxy radicals attached to the alkyl radical, that is, to form monoalkoxyalkyl and dialkoxyalkyl radicals. Alkoxyalkyl radicals can be “lower alkoxyalkyl” radicals having one to six carbon atoms and one or two alkoxy radicals. Examples of such radicals include methoxymethyl, methoxyethyl, ethoxyethyl, methoxybutyl and metoxypropyl. The alkyl in said “alkoxyalkyl” can be substituted with one or more of hydroxy, primary amine, carboxy, acid chloride, sulfonyl chloride, sulphonate, nitro, cyano, isothiocyanate, halogen, phosphonyl, sulphonyl, sulfamyl, carbonyl, and thiolyl. When e.g. the above “alkoxyl” or “alkoxyalkyl” radicals are substituted with one or more halo atoms, such as fluoro, chloro or bromo, “haloalkoxy” or “haloalkoxyalkyl” radicals are provided. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one, two or three rings wherein such rings may be attached together in a pendent manner or may be fused. When substituted, “aryl” can comprise one or more radicals selected from the group of radicals consisting of hydroxy, primary amine, carboxy, acid chloride, sulfonyl chloride, sulphonate, nitro, cyano, isothiocyanate, halogen, phosphonyl, sulphonyl, sulfamyl, carbonyl, and thiolyl. Examples of “aryl” include aromatic radicals such as phenyl, pentafluorphenyl, naphthyl, tetrahydronaphthyl, indane and biphenyl.

The term “heterocyclic” embraces saturated, partially saturated and unsaturated heteroatom-containing ring-shaped radicals, where the heteroatoms may be selected from nitrogen, sulfur and oxygen. When substituted, “heterocyclic” can comprise one or more radicals selected from the group of radicals consisting of hydroxy, primary amine, carboxy, acid chloride, sulfonyl chloride, sulphonate, nitro, cyanc, isothiocyanate, halogen, phosphonyl, sulphonyl, sulfamyl, carbonyl, and thiolyl. Examples of saturated heterocyclic radicals include e.g. saturated 3 to 6-membered heteromonocylic group containing 1 to 4 nitrogen atoms [e.g. pyrrolidinyl, imidazolidinyl, piperidino, piperazinyl, etc.]; saturated 3 to 6-membered heteromonocyclic group containing 1 to 2 oxygen atoms and 1 to 3 nitrogen atoms [e.g. morpholinyl, etc.]; saturated 3 to 6-membered heteromonocyclic group containing 1 to 2 sulfur atoms and 1 to 3 nitrogen atoms [e.g. thiazolidinyl, etc.]. Examples of partially saturated heterocyclic radicals include dihydrothiophene, dihydropyran, dihydrofuran and dihydrothiazole.

The term “heteroaryl” embraces unsaturated heterocyclic radicals. When substituted, “heteroaryl” can comprise one or more radicals selected from the group of radicals consisting of hydroxy, primary amine, secondary amine, carboxy, acid chloride, sulfonyl chloride, sulphonate, nitro, cyano, isothiocyanate, halogen, phosphonyl, sulphonyl, sulfamyl, carbonyl, and thiolyl. Examples of unsaturated heterocyclic radicals, also termed “heteroaryl” radicals, include e.g. unsaturated 5 to 6 membered heteromonocyclic group containing 1 to 4 nitrogen atoms, for example, pyrrolyl, pyrrolinyl, imidazolyl, pyrazolyl 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl, etc.] tetrazolyl [e.g. 1H-tetrazolyl, 2H-tetrazolyl, etc.], etc.; unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo[1,5-b]pyridazinyl, etc.], etc.; unsaturated 3 to 6-membered heteromonocyclic group containing an oxygen atom, for example, pyranyl, 2-furyl, 3-furyl, etc.; unsaturated 5 to 6-membered heteromonocyclic group containing a sulfur atom, for example, 2-thienyl, 3-thienyl, etc.; unsaturated 5- to 6-membered heteromonocyclic group containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl, etc.] etc.; unsaturated condensed heterocyclic group containing 1 to 2 oxygen atoms and 1 to 3 nitrogen atoms [e.g. benzoxazolyl, benzoxadiazolyl, etc.]; unsaturated 5 to 6-membered heteromonocyclic group containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl, etc.] etc.; unsaturated condensed heterocyclic group containing 1 to 2 sulfur atoms and 1 to 3 nitrogen atoms [e.g., benzothiazolyl, benzothiadiazolyl, etc.] and the like. The term “heteroaryl” or “unsaturated heterocyclic radical” also embraces radicals where heterocyclic radicals are fused with aryl radicals. Examples of such fused bicyclic radicals include benzofuran, benzothiophene, and the like. Said “heterocyclic group” can be substituted with one or more radicals selected from the group of radicals consisting of hydroxy, primary amine, carboxy, acid chloride, sulfonyl chloride, sulphonate, nitro, cyano, isothiocyanate, halogen, phosphonyl, sulphonyl, sulfamyl, carbonyl, and thiolyl, said substitution generating a substituted “heteroaryl”, optionally a substituted “heteroaryl” fused with an “aryl” radical which can be substituted or unsubstituted. When substituted, the “aryl” is substituted as described herein above. Preferred heterocyclic radicals include five to ten membered fused or unfused radicals. More preferred examples or heteroaryl radicals include benzofuryl, 2,3-dihydrobenzofuryl, benzotrienyl, indolyl, dihydroindolyl, chromanyl, benzopyran, thiochromanyl, benzothiopyran, benzodioxolyl, benzodioxanyl, pyridyl, thienyl, thiazolyl, oxazolyl, furyl, and pyrazinyl.

The term “sulfonyl”, whether used alone or linked to other terms such as alkylsulfonyl, denotes respectively divalent radicals —SO₂—.

“Alkylsulfonyl” embraces alkyl radicals attached to a sulfonyl radical, where alkyl can be substituted is defined as above. Alkylsulfonyl radicals can be “lower alkylsulfonyl” radicals having one to six carbon atoms. Examples of such lower alkylsulfonyl radicals include methylsulfonyl, ethylsulfonyl and propylsulfonyl.

The term “arylsulfonyl” embraces aryl radicals as defined above, including substituted aryl radicals, attached to a sulfonyl radical. Examples of such radicals include phenylsulfonyl.

The terms “sulfamyl,” “aminosulfonyl” and “sulfonamidyl,” whether alone or used with terms such as “N-alkylaminosulfonyl”, “N-arylaminosulfonyl”, “N,N-dialkylaminosulfonyl” and “N-alkyl-N-arylaminosulfonyl”, denotes a sulfonyl radical substituted with an amine radical, forming a sulfonamide (—SO₂NH₂).

The terms “N-alkylaminosulfonyl” and “N,N-dialkylaminosulfonyl” denote sulfamyl radicals substituted respectively, with one alkyl radical, or two alkyl radicals, optionally substituted alkyl radicals as described herein above. Akylaminosulfonyl radicals can be “lower alkylaminosulfonyl” radicals having one to six carbon atoms. Examples of such lower alkylaminosulfonyl radicals include N-methylaminosulfonyl, N-ethylaminosulfonyl and N-methyl-N-ethylaminosulfonyl.

The terms “N-arylaminosulfonyl” and “N-alkyl-N-arylaminosulfonyl” denote sulfamyl radicals substituted, respectively, with one aryl radical, or one alkyl and one aryl radical, optionally substituted aryl and/or alkyl radicals as described herein above. N-alkyl-N-arylaminosulfonyl radicals can be “lower N-alkyl-N-arylsulfonyl” radicals having alkyl radicals of one to six carbon atoms. Examples of such lower N-alkyl-N-aryl aminosulfonyl radicals include N-methyl-phenylaminosulfonyl and N-ethylphenylaminosulfonyl.

The terms “carboxy” or “carboxyl”, whether used alone or with other terms, such as “carboxyalkyl”, denotes —CO₂H.

The term “carboxyalkyl” or “alkanoyl” embraces radicals having a carboxy radical as defined above, attached to an alkyl radical as described herein above. When substituted, the “alkyl” or “lower alkyl” can comprise one or more radicals selected from the group of radicals consisting of hydroxy, primary amine, carboxy, acid chloride, sulfonyl chloride, sulphonate, nitro, cyano, isothiocyanate, halogen, phosphonyl, sulphonyl, sulfamyl, carbonyl, and thiolyl. Examples of “carboxyalkyl” radicals include formyl, acetyl, propionyl (propanoyl), butanoyl (butyryl), isobutanoyl (isobutyryl), valeryl (pentanoyl), isovaleryl, pivaloyl, hexanoyl or the like.

The term “carbonyl”, whether used alone or with other terms, such as “alkylcarbonyl”, denotes —(C═O)—.

The term “alkylcarbonyl” embraces radicals having a carbonyl radical substituted with an alkyl radical. Alkylcarbonyl radicals can be “lower alkylcarbonyl” radicals having from one to six carbon atoms. Examples of such radicals include methylcarbonyl and ethylcarbonyl. When substituted, the “alkyl” or “lower alkyl” of the “alkylcarbonyl” can comprise one or more radicals selected from the group of radicals consisting of hydroxy, primary amine, carboxy, acid chloride, sulfonyl chloride, sulphonate, nitro, cyano, isothiocyanate, halogen, phosphonyl, sulphonyl, sulfamyl, and thiolyl.

The term “alkylcarbonylalkyl”, denotes an alkyl radical substituted with an “alkylcarbonyl” radical as described herein above. Both the alkyl and the alkylcarbonyl can be substituted as described herein above.

The term “alkoxycarbonyl” means a radical containing an alkoxy radical, as defined above, attached via an oxygen atom to a carbonyl radical. “Lower alkoxycarbonyl” embraces alkoxy radicals preferably having from one to six carbon atoms. Examples of “lower alkoxycarbonyl” ester radicals include substituted or unsubstituted methoxycarbonyl, ethoxycarbonyl, propoxycarbonyl, butoxycarbonyl and hexyloxycarbonyl.

The term “alkoxycarbonylalkyl” embraces radicals having “alkoxycarbonyl”, as defined above substituted to an optionally substituted alkyl radical. Alkoxycarbonylalkyl radicals can be “lower alkoxycarbonylalkyl” having lower alkoxycarbonyl radicals as defined above attached to one to six carbon atoms. Examples of such lower alkoxycarbonylalkyl radicals include methoxycarbonylmethyl, tert-butoxycarbonylethyl, and methoxycarbonylethyl.

The term “aminocarbonyl” when used by itself or with other terms such as “aminocarbonylalkyl”, “N-alkylaminocarbonyl”, “N-arylaminocarbonyl, “N,N-dialkylaminocarbonyl”, “N-alkyl-N-arylaminocarbonyl”, “N-alkyl-N-hydroxyaminocarbonyl” and “N-alkyl-N-hydroxyaminocarbonylalkyl”, denotes an amide group of the formula —C(═O)NH₂.

The terms “N-alkylaminocarbonyl” and “N,N-dialkylaminocarbonyl” denote aminocarbonyl radicals which have been substituted with one alkyl radical and with two alkyl radicals, respectively. The alkyl radicals can be substituted as described herein above. “Lower alkylaminocarbonyl” comprises lower alkyl radicals as described above attached to an aminocarbonyl radical.

The terms “N-arylaminocarbonyl” and “N-alkyl-N-arylaminocarbonyl” denote aminocarbonyl radicals substituted, respectively, with one aryl radical, or one alkyl and one aryl radical, wherein such radicals can be substituted as described herein above.

The term “aminocarbonylalkyl” embraces optionally substituted alkyl radicals substituted with aminocarbonyl radicals.

The term “N-cycloalkylaminocarbonyl” denotes aminocarbonyl radicals which have been substituted with at least one optionally substituted cycloalkyl radical. “Lower cycloalkylaminocarbonyl” comprises lower cycloalkyl radicals of three to seven carbon atoms, attached to an aminocarbonyl radical.

The term “aminoalkyl” embraces alkyl radicals substituted with one or more amino radicals. The alkyl radicals can be further substituted by one or more radicals selected from the group of radicals consisting of hydroxy, carboxy, acid chloride, sulfonyl chloride, sulphonate, nitro, cyano, isothiocyanate, halogen, phosphonyl, sulphonyl, sulfamyl, carbonyl, and thiolyl.

The term “alkylaminoalkyl” embraces aminoalkyl radicals having the nitrogen atom substituted with an optionally substituted alkyl radical.

The term “amidino” denotes an —C(═NH)—NH₂ radical.

The term “cyanoamidino” denotes an —C(═N—CN)—NH₂ radical.

The term “heterocyclicalkyl” embraces heterocyclic-substituted alkyl radicals. The alkyl radicals can themselves be substituted by one or more radicals selected from the group of radicals consisting of hydroxy, primary amino, carboxy, acid chloride, sulfonyl chloride, sulphonate, nitro, cyano, isothiocyanate, halogen, phosphonyl, sulphonyl, sulfamyl, carbonyl, and thiolyl. Heterocyclicalkyl radicals can be “lower heterocyclicalkyl” radicals preferably having from one to six carbon atoms and a heterocyclic radical. Examples include such radicals as pyrrolidinylmethyl, pyridylmethyl and thienylmethyl.

The term “aralkyl” embraces aryl-substituted alkyl radicals. The alkyl radicals can themselves be substituted by one or more radicals selected from the group of radicals consisting of hydroxy, primary amino, carboxy, acid chloride, sulfonyl chloride, sulphonate, nitro, cyano, isothiocyanate, halogen, phosphonyl, sulphonyl, sulfamyl, carbonyl, and thiolyl. Aralkyl radicals can be “lower aralkyl” radicals having aryl radicals attached to alkyl radicals having from one to six carbon atoms. Examples of such radicals include benzyl, diphenylmethyl, triphenylmethyl, phenylethyl and diphenylethyl. The aryl in said aralkyl may be additionally substituted with halo, alkyl, alkoxy, halkoalkyl and haloalkoxy. The terms benzyl and phenylmethyl are interchangeable.

The term “cycloalkyl” embraces radicals having three to ten carbon atoms. Cycloalkyl radicals can be “lower cycloalkyl” radicals having three to seven carbon atoms. Examples include radicals such as cyclopropyl, cyclobutyl, cyclopentyl, cyclohexyl and cycloheptyl. The “cycloalkyl” can optionally be substituted by one or more radicals selected from the group of radicals consisting of hydroxy, primary amine, carboxy, acid chloride, sulfonyl chloride, sulphonate, nitro, cyano, isothiocyanate, halogen, phosphonyl, sulphonyl, sulfamyl, carbonyl, and thiolyl.

The term “cycloalkenyl” embraces unsaturated cyclic radicals having three to ten carbon atoms. The “cycloalkenyl” can optionally be substituted by one or more radicals selected from the group of radicals consisting of hydroxy, primary amine, carboxy, acid chloride, sulfonyl chloride, sulphonate, nitro, cyano, isothiocyanate, halogen, phosphonyl, sulphonyl, sulfamyl, carbonyl, and thiolyl. Examples include cyclobutenyl, cyclopentenyl, cyclohexenyl and cycloheptenyl, which can optionally be substituted as described above.

The term “alkylthio” embraces radicals containing a linear or branched alkyl radical, of one to ten carbon atoms, attached to a divalent sulfur atom. An example of “alkylthio” is methylthio, (CH₃—S—). The alkyl radical can be substituted as described herein above.

The term “alkylsulfinyl” embraces radicals containing a linear or branched alkyl radical, of one to ten carbon atoms, attached to a divalent —S(═O)— atom. The alkyl radical can be substituted as described herein above.

The term “aminoalkyl” embraces alkyl radicals substituted with amino radicals. The alkyl radicals can be further substituted by one or more radicals selected from the group of radicals consisting of hydroxy, carboxy, acid chloride, sulfonyl chloride, sulphonate, nitro, cyano, isothiocyanate, halogen, phosphonyl, sulphonyl, sulfamyl, carbonyl, and thiolyl. Aminoalkyl radicals can be “lower aminoalkyl” having from one to six carbon atoms. Examples include aminomethyl, aminoethyl and aminobutyl which can optionally be further substituted as described above.

The term “alkylaminoalkyl” embraces aminoalkyl radicals having the nitrogen atom substituted with at least one alkyl radical. Alkylaminoalkyl radicals can be “lower alkylaminoalkyl” having one to six carbon atoms attached to a lower aminoalkyl radical as described above. The alkyl radical can be substituted as described herein above.

The terms “N-alkylamino” and “N,N-dialkylamino” denote amino groups which have been substituted with one alkyl radical and with two alkyl radicals, respectively. The alkyl radical can be substituted as described herein above. Alkylamino radicals can be “lower alkylamino” radicals having one or two alkyl radicals of one to six carbon atoms, attached to a nitrogen atom. Suitable “alkylamino” may be mono or dialkylamino such as N-methylamino, N-ethylamino, N,N-dimethylamino, N,N-diethylamino or the like.

The term “arylamino” denotes amino groups which have been substituted with one or two aryl radicals, such as N-phenylamino. The “arylamino” radicals may be further substituted on the aryl ring portion of the radical. Substitutions can include one or more of hydroxy, amino, carboxy, acid chloride, sulfonyl chloride, sulphonate, nitro, cyano, isothiocyanate, halogen, phosphonyl, sulphonyl, sulfamyl, carbonyl, and thiolyl.

The term “aralkylamino” denotes amino groups which have been substituted with one or two aralkyl radicals, such as N-benzylamino. The “aralkylamino” radicals may be further substituted on the aryl ring portion of the radical. Substitutions can include one or more of hydroxy, amino, carboxy, acid chloride, sulfonyl chloride, sulphonate, nitro, cyano, isothiocyanate, halogen, phosphonyl, sulphonyl, sulfamyl, carbonyl, and thiolyl.

The terms “N-alkyl-N-arylamino” and “N-aralkyl-N-alkylamino” denote amino groups which have been substituted with one aralkyl and one alkyl radical, or one aryl and one alkyl radical, respectively, to an amino group. The aralkyl and/or alkyl and/or aryl radicals can be substituted as described herein above.

The terms “N-arylaminoalkyl” and “N-aralkylaminoalkyl” denote amino groups which have been substituted with one aryl radicals or one aralkyl radical, respectively, and having the amino group attached to an alkyl radical. The aralkyl and/or alkyl and/or aryl radicals can be substituted as described herein above. Arylaminoalkyl radicals can be “lower arylaminoalkyl” having the arylamino radical attached to one to six carbon atoms. Examples of such radicals include N-phenylaminomethyl and N-phenyl-N-methylaminomethyl.

The terms “N-alkyl-N-arylaminoalkyl”, and “N-aralkyl-N-alkylaminoalkyl” denote N-alkyl-N-arylamino and N-alkyl-N-aralkylamino groups, respectively, and having the amino group attached to alkyl radicals which can be substituted as described herein above.

The term “acyl”, whether used alone, or within a term such as “acylamino”, denotes a radical provided by the residue after removal of hydroxyl from an organic acid.

The term “acylamino” embraces an amino radical substituted with an acyl group. An examples of an “acylamino” radical is acetylamino or acetamido (CH₃C(═O)—NH—) where the amine may be further substituted with alkyl, aryl or aralkyl, wherein said alkyl, aryl or aralkyl can be substituted as described herein above.

The term “arylthio” embraces aryl radicals of six to ten carbon atoms, attached to a divalent sulfur atom. The aryl can be substituted as described herein above. An example of “arylthio” is phenylthio.

The term “aralkylthio” embraces aralkyl radicals as described above, attached to a divalent sulfur atom. The aralkyl radicals can be further substituted as described herein above. An example of “aralkylthio” is benzylthio.

The term “aryloxy” embraces aryl radicals, as defined above, attached to an oxygen atom. The aryl can be substituted as described herein above. Examples of such radicals include phenoxy.

The term “aralkoxy” embraces oxy-containing aralkyl radicals attached through an oxygen atom to other radicals. The aralkyl can be substituted as described herein above. Aralkoxy radicals can be “lower aralkoxy” radicals having phenyl radicals attached to lower alkoxy radical as described above.

The term “haloaralkyl” embraces aryl radicals as defined above attached to haloalkyl radicals. The aryl can be further substituted as described herein above.

The term “carboxyhaloalkyl” embraces carboxyalkyl radicals as defined above having halo radicals attached to the alkyl portion. The alkyl portion can be further substituted as described herein above.

The term “alkoxycarbonylhaloalkyl” embraces alkoxycarbonyl radicals as defined above substituted on a haloalkyl radical. The haloalkyl radical can be further substituted by one or more of hydroxy, amino, carboxy, acid chloride, sulfonyl chloride, sulphonate, nitro, cyano, isothiocyanate, phosphonyl, sulphonyl, sulfamyl, carbonyl, and thiolyl.

The term “aminocarbonylhaloalkyl” embraces aminocarbonyl radicals as defined above substituted on an optionally substituted haloalkyl radical wherein the alkyl is substituted by one or more of hydroxy, amino, carboxy, acid chloride, sulfonyl chloride, sulphonate, nitro, cyano, isothiocyanate, phosphonyl, sulphonyl, sulfamyl, carbonyl, and thiolyl.

The term “alkylaminocarbonylhaloalkyl” embraces alkylaminocarbonyl radicals as defined above substituted on an optionally substituted haloalkyl radical as described above.

The term “alkoxycarbonylcyanoalkenyl” embraces alkoxycarbonyl radicals as defined above, and a cyano radical, both substituted on an optionally substituted alkenyl radical.

The term “carboxyalkylaminocarbonyl” embraces aminocarbonyl radicals substituted with carboxyalkyl radicals, as defined above. The carboxyalkyl can be further substituted. Substitutions can include one or more of hydroxy, amino, acid chloride, sulfonyl chloride, sulphonate, nitro, cyano, isothiocyanate, halogen, phosphonyl, sulphonyl, sulfamyl, carbonyl, and thiolyl.

The term “aralkoxycarbonylalkylaminocarbonyl” embraces aminocarbonyl radicals substituted with aryl-substituted alkoxycarbonyl radicals, as defined above.

The term “cycloalkylalkyl” embraces cycloalkyl radicals having three to ten carbon atoms attached to an alkyl radical, as defined above. Cycloalkylalkyl radicals can be “lower cycloalkylalkyl” radicals having cycloalkyl radicals attached to lower alkyl radicals as defined above. Examples include radicals such as cyclopropylmethyl, cyclobutylmethyl, and cyclohexylethyl.

The term “aralkenyl” embraces optionally substituted aryl radicals attached to alkenyl radicals having two to ten carbon atoms, such as phenylbutenyl, and phenylethenyl or styryl. When substituted the aryl can be substituted with one or more of hydroxy, amino, carboxy, acid chloride, sulfonyl chloride, sulphonate, nitro, cyano, isothiocyanate, halogen, phosphonyl, sulphonyl, sulfamyl, carbonyl, and thiolyl.

The term “Pol” denotes a polymerized substituted or unsubstituted styrene, a polymerized substituted or unsubstituted acrylate, a polymerized substituted or unsubstituted (meth)acrylate, a polymerized substituted or unsubstituted hydroxymethyl(meth)acrylate; a polymerized substituted or unsubstituted acrylamide, a polymerized substituted or unsubstituted vinylacetate; a polymerized substituted or unsubstituted vinylether; a polymerized substituted or unsubstituted vinylpyrrolidone, a polymerized substituted or unsubstituted oxirane; a polymerized substituted or unsubstituted oxetane, a polymerized substituted or unsubstituted oxolane; a polymerized substituted or unsubstituted episulfide; a polymerized substituted or unsubstituted thiotane; a polymerized substituted or unsubstituted cyclic amine.

The term “derivative of a reactive group” denotes a reactive group (e.g. a reactive group of a substituted alkyl) which has been reacted with a chemical moiety, said reaction generating the derivative by attaching the chemical moiety (optionally the part of the chemical moiety which is not consumed in the reaction with the reactive group) to the fluorescent dye compound. The derivative can be e.g. an activated ester or a monomer or a macromonomer.

The term “reactive moiety” is used interchangeably with “derivative of a reactive group”. A substituent of the fluorescent dye compound can comprise a “reactive moiety”, or the “reactive moiety” can be attached to the fluorescent dye compound by reaction with a substituent in the form of e.g. a substituted alkyl capable of reacting with the reactive moiety (precursor).

### DETAILED DESCRIPTION OF THE INVENTION

Aspects and preferred embodiments of the present invention are described herein below.

In one embodiment, there is provided a fluorescent dye compound comprising the structure

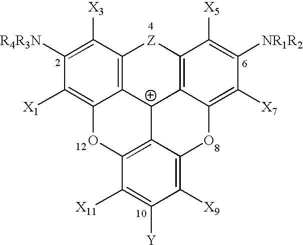

Preferably, the compound comprises one or more substituents in either one or more of the ortho positions (positions 1, 3, 5, 7, 9 and 11) and/or one or more of the para positions (positions 2, 6 and 10), more preferably one or more substituents in one or more of the para positions.

The para positions (2, 6 and 10): In order to obtain intense absorbance it is preferred that at least two of these positions carry a substituted amino group. In one embodiment, the invention is directed to compounds wherein position 10 is unsubstituted.

The ortho positions (1, 3, 5, 7, 9, 11): These positions can optionally be substituted with one or more substituents. In general, electronegative substituents should give raise to a redshift, while electropositive groups should give a blueshift. Since steric interactions between any group in these positions and the amino groups may lead to decreased fluorescence quantum yields, the most relevant substituents for these positions are expected to be F and Cl which could be introduced in order to redshift the absorbance/emission maximum.

Substituents comprising reactive groups: In general, it is preferred to introduce substituents comprising reactive groups via one of the para amino groups. However, the substituents comprising reactive groups can also be attached in an ortho position. Ortho- and para-positions are indicated herein above.

In a main aspect, the invention relates to a fluorescent dye compound comprising the structure 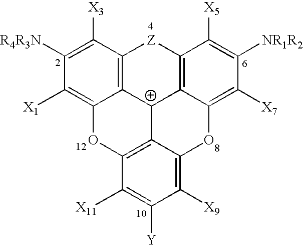
wherein X₁, X₃, X₅, X₇, X₉, and X₁₁ are independently H, Cl or F;
wherein Y is selected from the group consisting of H, Cl, F, NR₅R₆, OR₇, SR₈, and R₉;
wherein Z is O or NR₁₀, and
wherein R₁, R₂, R₃, R₄, R₅, R₆, R₇, R₈, R₉, and R₁₀ is independently H, an optionally substituted alkyl, an optionally substituted aryl, or an optionally polymerized substituted or unsubstituted monomer selected from the group consisting of styrene, acrylate, (meth)acrylate, hydroxymethyl(meth)acrylate, acrylamide, acetate, vinylacetate, vinylether, vinylpyrrolidone, oxirane, oxetane, oxolane, episulfide, thiotane, cyclic amine, or R₁ and R₂ together with the nitrogen to which they are attached form a substituted or unsubstituted heterocyclic, or R₃ and R₄ together with the nitrogen to which they are attached form a substituted or unsubstituted heterocyclic, or R₅ and R₆ together with the nitrogen to which they are attached form a substituted or unsubstituted heterocyclic, and/or wherein at least one of R₁, R₂, R₃, R₄, R₅, R₆, R₇, R₈, R₉, and R₁₀ comprises at least one reactive group or at least one reactive moiety,
wherein the at least one reactive group is selected from the group consisting of vinyl, allyl, hydroxy, primary amine, secondary amine, carboxy, carbonyl, nitro, cyano, isothiocyanate, halogen, phosphonyl, sulphonate, sulphonyl, sulfamyl, and thiolyl, including any combination thereof,
wherein the at least one reactive moiety is selected from the group consisting of an activated ester, such as N-succinimidyl ester, maleimide ester, or fluorophenol ester, such as pentafluorophenol ester; acid halide, such as acid chloride or acid bromide; sulfonyl halide, such as sulfonyl chloride or sulfonyl bromide; tosylate, mesylate, phtalimido, azido, an optionally polymerized substituted or unsubstituted styrene, an optionally polymerized substituted or unsubstituted acrylate, an optionally polymerized substituted or unsubstituted (meth)acrylate, an optionally polymerized substituted or unsubstituted hydroxymethyl(meth)acrylate; an optionally polymerized substituted or unsubstituted acrylamide, an optionally polymerized substituted or unsubstituted acetate, an optionally polymerized substituted or unsubstituted vinylacetate; an optionally polymerized substituted or unsubstituted vinylether; an optionally polymerized substituted or unsubstituted vinylpyrrolidone, an optionally polymerized substituted or unsubstituted oxirane; an optionally polymerized substituted or unsubstituted oxetane, an optionally polymerized substituted or unsubstituted oxolane; an optionally polymerized substituted or unsubstituted episulfide; an optionally polymerized substituted or unsubstituted thiotane; and an optionally polymerized substituted or unsubstituted cyclic amine,
with the proviso that R₁ to R₆ are not all identical linear alkyls when Y is NR₅R₆ and Z is O, and that R₁ and R₂ are not both ethyl when NR₃R₄ and NR₅R₆ both constitute a morpholinyl ring.

In a preferred embodiment of the fluorescent dye compound of the invention, X₁, X₃, X₅, X₇, X₉, and X₁₁ , are all H. In another preferred embodiment, X₁, X₃, X₅, X₇, X₉, and X₁₁ , are all Cl. In a yet other embodiment, X₁, X₃, X₅, X₇, X₉, and X₁₁ are all F.

In a further preferred embodiment, Y is selected from H, Cl, and F. Thus, in a preferred embodiment Y is H, in another preferred embodiment Y is Cl, and in a yet other preferred embodiment, Y is F.

In a highly preferred embodiment, X is one of the above described and Y is one of the above described.

In a further preferred embodiment of the fluorescent dye compound of the invention, Y is F and Z is O or NR₁₀. Thus, in one preferred embodiment, Y is F and Z is O and in another preferred embodiment, Y is F and Z is NR₁₀.

In further preferred embodiment, Y is selected from the group consisting of NR₅R₆, OR₇, SR₈, and R₉, wherein Y more preferably is NR₅R₆.

In a highly further preferred embodiment, Y is NR₅R₆ and Z is O or NR₁₀.

In a further more preferred embodiment, Y is NR₅R₆, Z is NR₁₀ and each of R₁, R₂, R₃, R₄, R₅, R₆ is a substituted or unsubstituted alkyl, or R₁ and R₂ together with the nitrogen to which they are attached form a substituted or unsubstituted heterocyclic, or R₃ and R₄ together with the nitrogen to which they are attached form a substituted or unsubstituted heterocyclic, or R₅ and R₆ together with the nitrogen to which they are attached form a substituted or unsubstituted heterocyclic, and R₁₀ is an optionally polymerized substituted or unsubstituted monomer selected from the group consisting of styrene, acrylate, (meth)acrylate, hydroxymethyl(meth)acrylate, acrylamide, acetate, vinylacetate, vinylether, vinylpyrrolidone, oxirane, oxetane, oxolane, episulfide, thiotane, and cyclic amine.

In a further more preferred embodiment, Y is NR₅R₆, Z is NR₁₀ and each of R₁, R₂, R₃, R₄, R₅, R₆ is a substituted or unsubstituted alkyl, or R₁ and R₂ together with the nitrogen to which they are attached form a substituted or unsubstituted heterocyclic, or R₃ and R₄ together with the nitrogen to which they are attached form a substituted or unsubstituted heterocyclic, or R₅ and R₆ together with the nitrogen to which they are attached form a substituted or unsubstituted heterocyclic, and R₁₀ comprises a reactive moiety selected from the group consisting of an activated ester, such as N-succinimidyl ester, maleimide ester, or fluorophenol ester, such as pentafluorophenol ester; acid halide, such as acid chloride or acid bromide; sulfonyl halide, such as sulfonyl chloride or sulfonyl bromide; tosylate, mesylate, phtalimido, and azido.

In even more preferred embodiments of the two latter more preferred embodiments above, 1 to 3 of R₁ to R₆ are substituted alkyl.

In a further preferred embodiment, Y is NR₅R₆, Z is O and each of R₁, R₂, R₃, R₄, R₅ is a substituted or unsubstituted alkyl, or R₁ and R₂ together with the nitrogen to which they are attached form a substituted or unsubstituted heterocyclic, or R₃ and R₄ together with the nitrogen to which they are attached form a substituted or unsubstituted heterocyclic, and R₆ is an optionally polymerized substituted or unsubstituted monomer selected from the group consisting of styrene, acrylate, (meth)acrylate, hydroxymethyl(meth)acrylate, acrylamide, acetate, vinylacetate, vinylether, vinylpyrrolidone, oxirane, oxetane, oxolane, episulfide, thiotane, and cyclic amine.

In a further preferred embodiment, Y is NR₅R₆, Z is O and each of R₁, R₂, R₃, R₄, R₅ is a substituted or unsubstituted alkyl, or R₁ and R₂ together with the nitrogen to which they are attached form a substituted or unsubstituted heterocyclic, or R₃ and R₄ together with the nitrogen to which they are attached form a substituted or unsubstituted heterocyclic, and R₆ comprises a reactive moiety selected from the group consisting of an activated ester, such as N-succinimidyl ester, maleimide ester, or fluorophenol ester, such as pentafluorophenol ester; acid halide, such as acid chloride or acid bromide; sulfonyl halide, such as sulfonyl chloride or sulfonyl bromide; tosylate, mesylate, phtalimido, and azido.

In even more preferred embodiments of the two latter more preferred embodiments above, from 1 to 3 of R₁ to R₅ are substituted alkyl.

In a further preferred embodiment, Y is NR₅R₆, Z is O and each of R₁, R₂, R₃, R₅ is a substituted or unsubstituted alkyl, or R₁ and R₂ together with the nitrogen to which they are attached form a substituted or unsubstituted heterocyclic, and R₅ and R₆ are independently an optionally polymerized substituted or unsubstituted monomer selected from the group consisting of styrene, acrylate, (meth)acrylate, hydroxymethyl-(meth)acrylate, acrylamide, acetate, vinylacetate, vinylether, vinylpyrrolidone, oxirane, oxetane, oxolane, episulfide, thiotane, and cyclic amine, wherein R₅ and R₆ can be different or the same.

In a further preferred embodiment, Y is NR₅R₆, Z is O and each of R₁, R₂, R₃, R₅ is a substituted or unsubstituted alkyl, or R₁ and R₂ together with the nitrogen to which they are attached form a substituted or unsubstituted heterocyclic, and each of R₅ and R₆ comprises a reactive moiety selected from the group consisting of an activated ester, such as N-succinimidyl ester, maleimide ester, or fluorophenol ester, such as pentafluorophenol ester; acid halide, such as acid chloride or acid bromide; sulfonyl halide, such as sulfonyl chloride or sulfonyl bromide; tosylate, mesylate, phtalimido, and azido, wherein R₅ and R₆ can be different or the same.

In even more preferred embodiments of the two latter more preferred embodiments above, from 1 to 3 of R₁, R₂, R₃, R₅ are substituted alkyl.

In a further preferred embodiment, Y is NR₅R₆, Z is O, and each of R₁, R₃, R₅ is a substituted or unsubstituted alkyl, and R₂, R₄ and R₆ is independently an optionally polymerized substituted or unsubstituted monomer selected from the group consisting of styrene, acrylate, (meth)acrylate, hydroxymethyl(meth)acrylate, acrylamide, acetate, vinylacetate, vinylether, vinylpyrrolidone, oxirane, oxetane, oxolane, episulfide, thiotane, and cyclic amine, wherein R₅ and R₆ can be different or the same.

In a further preferred embodiment, Y is NR₅R₆, Z is O, and each of R₁, R₃, R₅ is a substituted or unsubstituted alkyl, and each of R₂, R₄ and R₆ comprises a reactive moiety selected from the group consisting of an activated ester, such as N-succinimidyl ester, maleimide ester, or fluorophenol ester, such as pentafluorophenol ester; acid halide, such as acid chloride or acid bromide; sulfonyl halide, such as sulfonyl chloride or sulfonyl bromide; tosylate, mesylate, phtalimido, and azido, wherein R₂, R₄ and R₆ can be different or the same.

In even more preferred embodiments of the two latter more preferred embodiments above, from 1 to all of R₁, R₃, R₅ are substituted alkyl.

In a further preferred embodiment of the fluorescent dye of the invention, Y is OR₇. In a even further preferred embodiment, Y is OR₇ and Z is O or NR₁₀. Thus, in one preferred embodiment, Y is OR₇ and Z is O and in another preferred embodiment, Y is OR₇ and Z is NR₁₀.

In a more preferred embodiment, Y is OR₇, Z is O, and each of R₁, R₂, R₃, R₄ is a substituted or unsubstituted alkyl, or R₁ and R₂ together with the nitrogen to which they are attached form a substituted or unsubstituted heterocyclic, or R₃ and R₄ together with the nitrogen to which they are attached form a substituted or unsubstituted heterocyclic, and R₇ is an optionally polymerized substituted or unsubstituted monomer selected from the group consisting of styrene, acrylate, (meth)acrylate, hydroxymethyl(meth)acrylate, acrylamide, acetate, vinylacetate, vinylether, vinylpyrrolidone, oxirane, oxetane, oxolane, episulfide, thiotane, and cyclic amine.

In a more preferred embodiment, Y is OR₇, Z is O, and each of R₁, R₂, R₃, R₄ is a substituted or unsubstituted alkyl, or R₁ and R₂ together with the nitrogen to which they are attached form a substituted or unsubstituted heterocyclic, or R₃ and R₄ together with the nitrogen to which they are attached form a substituted or unsubstituted heterocyclic, and R₇ comprises a reactive moiety selected from the group consisting of an activated ester, such as N-succinimidyl ester, maleimide ester, or fluorophenol ester, such as pentafluorophenol ester; acid halide, such as acid chloride or acid bromide; sulfonyl halide, such as sulfonyl chloride or sulfonyl bromide; tosylate, mesylate, phtalimido, and azido.

In even more preferred embodiments of the two latter more preferred embodiments above, from 1 to 3 of R₁, R₂, R₃, R₄ are substituted alkyl.

In a further more preferred embodiment, Y is OR₇, Z is NR₁₀, and each of R₁, R₂, R₃, R₄ is a substituted or unsubstituted alkyl, or R₁ and R₂ together with the nitrogen to which they are attached form a substituted or unsubstituted heterocyclic, or R₃ and R₄ together with the nitrogen to which they are attached form a substituted or unsubstituted heterocyclic, and R₇ and R₁₀ is independently an optionally polymerized substituted or unsubstituted monomer selected from the group consisting of styrene, acrylate, (meth)acrylate, hydroxymethyl(meth)acrylate, acrylamide, acetate, vinylacetate, vinylether, vinylpyrrolidone, oxirane, oxetane, oxolane, episulfide, thiotane, and cyclic amine, wherein R₇ and R₁₀ can be different or the same.

In a further more preferred embodiment, Y is OR₇, Z is NR₁₀, and each of R₁, R₂, R₃, R₄ is a substituted or unsubstituted alkyl, or R₁ and R₂ together with the nitrogen to which they are attached form a substituted or unsubstituted heterocyclic, or R₃ and R₄ together with the nitrogen to which they are attached form a substituted or unsubstituted heterocyclic, and each of R₇ and R₁₀ comprises a reactive moiety selected from the group consisting of an activated ester, such as N-succinimidyl ester, maleimide ester, or fluorophenol ester, such as pentafluorophenol ester; acid halide, such as acid chloride or acid bromide; sulfonyl halide, such as sulfonyl chloride or sulfonyl bromide; tosylate, mesylate, phtalimido, and azido, wherein R₇ and R₁₀ can be different or the same.

In even more preferred embodiments of the two latter more preferred embodiments above, from 1 to 3 of R₁, R₂, R₃, R₄ are substituted alkyl.

In a further preferred embodiment of the fluorescent dye of the invention, Y is SR₈.

In a even further preferred embodiment, Y is SR₈ and Z is O or NR₁₀. Thus, in one preferred embodiment, Y is SR₈ and Z is O and in another preferred embodiment, Y is SR₈ and Z is NR₁₀.

In a more preferred embodiment, Y is SR₈, Z is O, and each of R₁, R₂, R₃, R₄ is a substituted or unsubstituted alkyl, or R₁ and R₂ together with the nitrogen to which they are attached form a substituted or unsubstituted heterocyclic, or R₃ and R₄ together with the nitrogen to which they are attached form a substituted or unsubstituted heterocyclic, and R₈ is an optionally polymerized substituted or unsubstituted monomer selected from the group consisting of styrene, acrylate, (meth)acrylate, hydroxymethyl(meth)acrylate, acrylamide, acetate, vinylacetate, vinylether, vinylpyrrolidone, oxirane, oxetane, oxolane, episulfide, thiotane, and cyclic amine.

In a more preferred embodiment, Y is SR₈, Z is O, and each of R₁, R₂, R₃, R₄ is a substituted or unsubstituted alkyl, or R₁ and R₂ together with the nitrogen to which they are attached form a substituted or unsubstituted heterocyclic, or R₃ and R₄ together with the nitrogen to which they are attached form a substituted or unsubstituted heterocyclic, and R₈ comprises a reactive moiety selected from the group consisting of an activated ester, such as N-succinimidyl ester, maleimide ester, or fluorophenol ester, such as pentafluorophenol ester; acid halide, such as acid chloride or acid bromide; sulfonyl halide, such as sulfonyl chloride or sulfonyl bromide; tosylate, mesylate, phtalimido, and azido.

In even more preferred embodiments of the two latter more preferred embodiments above, from 1 to 3 of R₁, R₂, R₃, R₄ are substituted alkyl.

In a more preferred embodiment, Y is SR₈, Z is NR₁₀, and each of R₁, R₂, R₃, R₄ is a substituted or unsubstituted alkyl, or R₁ and R₂ together with the nitrogen to which they are attached form a substituted or unsubstituted heterocyclic, or R₃ and R₄ together with the nitrogen to which they are attached form a substituted or unsubstituted heterocyclic, and R₈ and R₁₀ is independently an optionally polymerized substituted or unsubstituted monomer selected from the group consisting of styrene, acrylate, (meth)acrylate, hydroxymethyl(meth)acrylate, acrylamide, acetate, vinylacetate, vinylether, vinylpyrrolidone, oxirane, oxetane, oxolane, episulfide, thiotane, and cyclic amine, wherein R₈ and R₁₀ can be different or the same.

In a more preferred embodiment, Y is SR₈, Z is NR₁₀, and each of R₁, R₂, R₃, R₄ is a substituted or unsubstituted alkyl, or R₁ and R₂ together with the nitrogen to which they are attached form a substituted or unsubstituted heterocyclic, or R₃ and R₄ together with the nitrogen to which they are attached form a substituted or unsubstituted heterocyclic, and each of R₈ and R₁₀ comprises a reactive moiety selected from the group consisting of an activated ester, such as N-succinimidyl ester, maleimide ester, or fluorophenol ester, such as pentafluorophenol ester; acid halide, such as acid chloride or acid bromide; sulfonyl halide, such as sulfonyl chloride or sulfonyl bromide; tosylate, mesylate, phtalimido, and azido, wherein R₈ and R₁₀ can be different or the same.

In even more preferred embodiments of the two latter more preferred embodiments above, from 1 to 3 of R₁, R₂, R₃, R₄ are substituted alkyl.

In a further preferred embodiment of the fluorescent dye of the invention, Y is R₉.

In a even further preferred embodiment, Y is R₉ and Z is O or NR₁₀. Thus, in one preferred embodiment, Y is R₉ and Z is O and in another preferred embodiment, Y is R₉ and Z is NR₁₀.

In a even further preferred embodiment, Y is R₉, Z is O and each of R₁, R₂, R₃, R₄ is a substituted or unsubstituted alkyl, or R₁ and R₂ together with the nitrogen to which they are attached form a substituted or unsubstituted heterocyclic, or R₃ and R₄ together with the nitrogen to which they are attached form a substituted or unsubstituted heterocyclic, and R₉ is an optionally polymerized substituted or unsubstituted monomer selected from the group consisting of styrene, acrylate, (meth)acrylate, hydroxymethyl(meth)acrylate, acrylamide, acetate, vinylacetate, vinylether, vinylpyrrolidone, oxirane, oxetane, oxolane, episulfide, thiotane, and cyclic amine.

In a even further preferred embodiment, Y is R₉, Z is O and each of R₁, R₂, R₃, R₄ is a substituted or unsubstituted alkyl, or R₁ and R₂ together with the nitrogen to which they are attached form a substituted or unsubstituted heterocyclic, or R₃ and R₄ together with the nitrogen to which they are attached form a substituted or unsubstituted heterocyclic, and R₉ comprises a reactive moiety selected from the group consisting of an activated ester, such as N-succinimidyl ester, maleimide ester, or fluorophenol ester, such as pentafluorophenol ester; acid halide, such as acid chloride or acid bromide; sulfonyl halide, such as sulfonyl chloride or sulfonyl bromide; tosylate, mesylate, phtalimido, and azido.

In even more preferred embodiments of the two latter more preferred embodiments above, from 1 to 3 of R₁, R₂, R₃, R₄ are substituted alkyl.

In a even further preferred embodiment, Y is R₉, Z is NR₁₀, and each of R₁, R₂, R₃, R₄ is a substituted or unsubstituted alkyl, or R₁ and R₂ together with the nitrogen to which they are attached form a substituted or unsubstituted heterocyclic, or R₃ and R₄ together with the nitrogen to which they are attached form a substituted or unsubstituted heterocyclic, and R₉ and R₁₀ is independently an optionally polymerized substituted or unsubstituted monomer selected from the group consisting of styrene, acrylate, (meth)acrylate, hydroxymethyl(meth)acrylate, acrylamide, acetate, vinylacetate, vinylether, vinylpyrrolidone, oxirane, oxetane, oxolane, episulfide, thiotane, and cyclic amine, wherein R₉ and R₁₀ can be different or the same.

In a even further preferred embodiment, Y is R₉, Z is NR₁₀ and each of R₁, R₂, R₃, R₄ is a substituted or unsubstituted alkyl, or R₁ and R₂ together with the nitrogen to which they are attached form a substituted or unsubstituted heterocyclic, or R₃ and R₄ together with the nitrogen to which they are attached form a substituted or unsubstituted heterocyclic, and each of R₉ and R₁₀ comprises a reactive moiety selected from the group consisting of an activated ester, such as N-succinimidyl ester, maleimide ester, or fluorophenol ester, such as pentafluorophenol ester; acid halide, such as acid chloride or acid bromide; sulfonyl halide, such as sulfonyl chloride or sulfonyl bromide; tosylate, mesylate, phtalimido, and azido, wherein R₉ and R₁₀ can be different or the same.

In even more preferred embodiments of the two latter more preferred embodiments above, from 1 to 3 of R₁, R₂, R₃, R₄ are substituted alkyl.

In a highly preferred embodiment, the alkyl in the preferred embodiments described above, is a lower alkyl.

In a further highly preferred embodiment, R₁, R₂, R₃, R₄, R₅, and R₆, are independently H, an optionally substituted alkyl, or an optionally substituted aryl, and R₇, R₈, R₉, and R₁₀ are independently H, an optionally substituted alkyl, an optionally substituted aryl, or an optionally polymerized substituted or unsubstituted monomer selected from the group consisting of styrene, acrylate, (meth)acrylate, hydroxymethyl(meth)acrylate, acrylamide, acetate, vinylacetate, vinylether, vinylpyrrolidone, oxirane, oxetane, oxolane, episulfide, thiotane, cyclic amine, wherein at least one of R₇, R₈, R₉, and R₁₀ comprises at least one reactive group or at least one reactive moiety.

In another aspect, the fluorescent dye compound of the invention comprises the structure 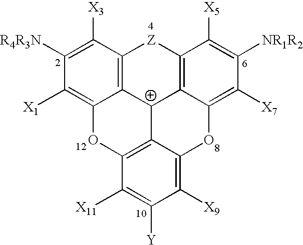
wherein X₁, X₃, X_{S}, X₇, X₉, and X₁₁ are independently H, Cl or F;
wherein Y is selected from the group consisting of H, Cl, F, NR₅R₆, OR₇, SR₈, and R₉;
wherein Z is O or NR₁₀, and
wherein substituents R₁ to R₁₀ are preferably selected independently from the group consisting of:
hydrido,
substituted and unsubstituted alkyl, substituted and unsubstituted haloalkyl, substituted and unsubstituted hydroxyalkyl, substituted and unsubstituted alkylsulfonyl,
substituted and unsubstituted alkenyl,
halo,
substituted and unsubstituted alkoxy, substituted and unsubstituted alkoxyalkyl, substituted and unsubstituted haloalkoxy, substituted and unsubstituted haloalkoxyalkyl,
substituted and unsubstituted aryl,
substituted and unsubstituted heterocyclic,
substituted and unsubstituted heteroaryl,
sulfonyl, substituted and unsubstituted alkylsulfonyl, substituted and unsubstituted arylsulfonyl, sulfamyl, sulfonamidyl, aminosulfonyl, substituted and unsubstituted N-alkylaminosulfonyl, substituted and unsubstituted N-arylaminosulfonyl, substituted and unsubstituted N,N-dialkylaminosulfonyl, substituted and unsubstituted N-alkyl-N-arylaminosulfonyl, substituted and unsubstituted N-alkylaminosulfonyl, substituted and unsubstituted N,N-dialkylaminosulfonyl, substituted and unsubstituted N-arylaminosulfonyl, substituted and unsubstituted N-alkyl-N-arylaminosulfonyl,
carboxy, substituted and unsubstituted carboxyalkyl,
carbonyl, substituted and unsubstituted alkylcarbonyl, substituted and unsubstituted alkylcarbonylalkyl,
substituted and unsubstituted alkoxycarbonyl, substituted and unsubstituted alkoxycarbonylalkyl,
aminocarbonyl, substituted and unsubstituted aminocarbonylalkyl, substituted and unsubstituted N-alkylaminocarbonyl, substituted and unsubstituted N-arylaminocarbonyl, substituted and unsubstituted N,N-dialkylaminocarbonyl, substituted and unsubstituted N-alkyl-N-arylaminocarbonyl, substituted and unsubstituted N-alkyl-N-hydroxyaminocarbonyl, substituted and unsubstituted N-alkyl-N-hydroxyaminocarbonylalkyl, substituted and unsubstituted N-alkylaminocarbonyl, substituted and unsubstituted N,N-dialkylaminocarbonyl, substituted and unsubstituted N-arylaminocarbonyl, substituted and unsubstituted N-alkyl-N-arylaminocarbonyl, substituted and unsubstituted aminocarbonylalkyl, substituted and unsubstituted N-cycloalkylaminocarbonyl,
substituted and unsubstituted aminoalkyl, substituted and unsubstituted alkylaminoalkyl,
amidino,
cyanoamidino,
substituted and unsubstituted heterocyclicalkyl,
substituted and unsubstituted aralkyl,
substituted and unsubstituted cycloalkyl,
substituted and unsubstituted cycloalkenyl,
substituted and unsubstituted alkylthio,
substituted and unsubstituted alkylsulfinyl,
substituted and unsubstituted N-alkylamino, substituted and unsubstituted N,N-dialkylamino,
substituted and unsubstituted arylamino, substituted and unsubstituted aralkylamino, substituted and unsubstituted N-alkyl-N-arylamino, substituted and unsubstituted N-aralkyl-N-alkylamino, substituted and unsubstituted N-arylaminoalkyl, substituted and unsubstituted N-aralkylaminoalkyl, substituted and unsubstituted N-alkyl-N-arylaminoalkyl, substituted and unsubstituted N-aralkyl-N-alkylaminoalkyl,
acyl, acylamino,
substituted and unsubstituted arylthio, substituted and unsubstituted aralkylthio,
substituted and unsubstituted aryloxy, substituted and unsubstituted aralkoxy,
substituted and unsubstituted haloaralkyl,
substituted and unsubstituted carboxyhaloalkyl,
substituted and unsubstituted alkoxycarbonylhaloalkyl, substituted and unsubstituted aminocarbonylhaloalkyl, substituted and unsubstituted alkylaminocarbonylhaloalkyl,
substituted and unsubstituted alkoxycarbonylcyanoalkenyl,
substituted and unsubstituted carboxyalkylaminocarbonyl,
substituted and unsubstituted aralkoxycarbonylalkylaminocarbonyl,
substituted and unsubstituted cycloalkylalkyl, and
substituted and unsubstituted aralkenyl,
wherein at least one of said substituents R1 to R10 comprises
a) one or more reactive groups selected from the group consisting of vinyl, allyl, hydroxy, primary amine, secondary amine, carboxy, carbonyl, nitro, cyano, isothiocyanate, halogen, phosphonyl, sulphonate, sulphonyl, sulfamyl, and thiolyl, or
b) one or more reactive moieties selected from the group consisting of an activated ester, such as N-succinimidyl ester, maleimide ester, or fluorophenol ester, including pentafluorophenol ester; acid halide, such as acid chloride or acid bromide; sulfonyl halide, such as sulfonyl chloride or sulfonyl bromide; tosylate, mesylate, phtalimido, azido, an optionally polymerized substituted or unsubstituted styrene, an optionally polymerized substituted or unsubstituted acrylate, an optionally polymerized substituted or unsubstituted (meth)acrylate, an optionally polymerized substituted or unsubstituted hydroxymethyl(meth)acrylate; an optionally polymerized substituted or unsubstituted acrylamide, an optionally polymerized substituted or unsubstituted acetate, an optionally polymerized substituted or unsubstituted vinylacetate; an optionally polymerized substituted or unsubstituted vinylether; an optionally polymerized substituted or unsubstituted vinylpyrrolidone, an optionally polymerized substituted or unsubstituted oxirane; an optionally polymerized substituted or unsubstituted oxetane, an optionally polymerized substituted or unsubstituted oxolane; an optionally polymerized substituted or unsubstituted episulfide; an optionally polymerized substituted or unsubstituted thiotane; and an optionally polymerized substituted or unsubstituted cyclic amine,
   with the proviso that R₁ to R₆ are not all identical linear alkyls when Y is NR₅R₆ and Z is O, and that R₁ and R₂ are not both ethyl when NR₃R₄ and NR₅R₆ both constitute a morpholinyl ring.

In a preferred embodiment, X₁, X₃, X_{S}, X₇, X₉, and X₁₁ are all H. In another preferred embodiment, X₁, X₃, X₅, X₇, X₉, and X₁₁ are all Cl. In a yet other preferred embodiment, X₁, X₃, X₅, X₇, X₉, and X₁₁ are all F.

In further preferred embodiments, Y is selected from H, Cl, and F. Thus, in one further preferred embodiment, Y is H. In another preferred embodiment, Y is Cl. And in another preferred embodiment, Y is F.

In further preferred embodiments, Y is selected from the group consisting of NR₅R₆, OR₇, SR₈, and R₉. Thus, in one further preferred embodiment, Y is NR₅R₆. In another preferred embodiment, Y is OR₇. In a yet other preferred embodiment, Y is SR₈. And in an even other preferred embodiment, Y is R₉.

In further preferred embodiments, Y is selected from the group consisting of NR₅R₆, OR₇, SR₈, and R₉, and Y comprises a reactive group capable of undergoing polymerization. The reactive group is preferably selected from the group consisting of vinyl, acrylate, methacrylate, acrylamide, methyl oxethane, ethyl oxethane, and ethylene oxide.

In further preferred embodiments, Y is selected from the group consisting of NR₅R₆, OR₇, SR₈, and R₉, and Y is a linker capable of linking the dye compound to a polymer matrix.

In further preferred embodiments, Y is selected from the group consisting of NR₅R₆, OR₇, SR₈, and R₉, and Y is selected from the group consisting of carboxylic acid, benzylic acid, N-succinimidyl ester, acid chloride, pentafluorophenol ester, tosylate, mesylate, halide, primary amine, sulfonyl chloride, isothiocyanate, maleimide, and thiol.

In further preferred embodiments, Y is selected from H, Cl, and F, and Z is O or NR₁₀. Thus, in one preferred embodiment, Y is selected from H, Cl, and F, and Z is O. In another preferred embodiment, Y is selected from H, Cl, and F, and Z is NR₁₀.

In further preferred embodiment, Y is selected from H, Cl, and F, and R₁ to R₁₀ are independently
hydrido,
substituted and unsubstituted alkyl, substituted and unsubstituted haloalkyl, substituted and unsubstituted hydroxyalkyl, substituted and unsubstituted alkylsulfonyl,
substituted and unsubstituted alkenyl,
halo,
substituted and unsubstituted alkoxy, substituted and unsubstituted alkoxyalkyl, substituted and unsubstituted haloalkoxy, substituted and unsubstituted haloalkoxyalkyl,
substituted and unsubstituted aryl,
substituted and unsubstituted heterocyclic,
substituted and unsubstituted heteroaryl,
sulfonyl, substituted and unsubstituted alkylsulfonyl, substituted and unsubstituted arylsulfonyl, sulfamyl, sulfonamidyl, aminosulfonyl, substituted and unsubstituted N-alkylaminosulfonyl, substituted and unsubstituted N-arylaminosulfonyl, substituted and unsubstituted N,N-dialkylaminosulfonyl, substituted and unsubstituted N-alkyl-N-arylaminosulfonyl, substituted and unsubstituted N-alkylaminosulfonyl, substituted and unsubstituted N,N-dialkylaminosulfonyl, substituted and unsubstituted N-arylaminosulfonyl, substituted and unsubstituted N-alkyl-N-arylaminosulfonyl,
carboxy, substituted and unsubstituted carboxyalkyl,
carbonyl, substituted and unsubstituted alkylcarbonyl, substituted and unsubstituted alkylcarbonylalkyl,
substituted and unsubstituted alkoxycarbonyl, substituted and unsubstituted alkoxycarbonylalkyl,
aminocarbonyl, substituted and unsubstituted aminocarbonylalkyl, substituted and unsubstituted N-alkylaminocarbonyl, substituted and unsubstituted N-arylaminocarbonyl, substituted and unsubstituted N,N-dialkylaminocarbonyl, substituted and unsubstituted N-alkyl-N-arylaminocarbonyl, substituted and unsubstituted N-alkyl-N-hydroxyaminocarbonyl, substituted and unsubstituted N-alkyl-N-hydroxyaminocarbonylalkyl, substituted and unsubstituted N-alkylaminocarbonyl, substituted and unsubstituted N,N-dialkylaminocarbonyl, substituted and unsubstituted N-arylaminocarbonyl, substituted and unsubstituted N-alkyl-N-arylaminocarbonyl, substituted and unsubstituted aminocarbonylalkyl, substituted and unsubstituted N-cycloalkylaminocarbonyl,
substituted and unsubstituted aminoalkyl, substituted and unsubstituted alkylaminoalkyl,
amidino,
cyanoamidino,
substituted and unsubstituted heterocyclicalkyl,
substituted and unsubstituted aralkyl,
substituted and unsubstituted cycloalkyl,
substituted and unsubstituted cycloalkenyl,
substituted and unsubstituted alkylthio,
substituted and unsubstituted alkylsulfinyl,
substituted and unsubstituted N-alkylamino, substituted and unsubstituted N,N-dialkylamino,
substituted and unsubstituted arylamino, substituted and unsubstituted aralkylamino, substituted and unsubstituted N-alkyl-N-arylamino, substituted and unsubstituted N-aralkyl-N-alkylamino, substituted and unsubstituted N-arylaminoalkyl, substituted and unsubstituted N-aralkylaminoalkyl, substituted and unsubstituted N-alkyl-N-arylaminoalkyl, substituted and unsubstituted N-aralkyl-N-alkylaminoalkyl,
acyl, acylamino,
substituted and unsubstituted arylthio, substituted and unsubstituted aralkylthio,
substituted and unsubstituted aryloxy, substituted and unsubstituted aralkoxy,
substituted and unsubstituted haloaralkyl,
substituted and unsubstituted carboxyhaloalkyl,
substituted and unsubstituted alkoxycarbonylhaloalkyl, substituted and unsubstituted aminocarbonylhaloalkyl, substituted and unsubstituted alkylaminocarbonylhaloalkyl,
substituted and unsubstituted alkoxycarbonylcyanoalkenyl,
substituted and unsubstituted carboxyalkylaminocarbonyl,
substituted and unsubstituted aralkoxycarbonylalkylaminocarbonyl,
substituted and unsubstituted cycloalkylalkyl, or
substituted and unsubstituted aralkenyl.

In another main aspect, the invention relates to a method for producing a polymer matrix comprising a fluorescent dye, said method comprising the steps of providing a monomer or a polymer matrix and reacting a fluorescent dye compound of the invention as describe herein, or a precursor thereof, with the monomer or polymer matrix, and optionally reacting the fluorescent dye compound precursor to obtain the fluorescent dye compound, and further optionally polymerizing the monomers to obtain a polymer matrix.

In a further aspect, the invention relates to the use of a fluorescent dye compound of the invention as described herein for visualising a beaded polymer matrix.

In an even further aspect, the invention relates to the use of a fluorescent dye compound of the invention as described herein in the synthesis of a beaded polymer matrix.

In a further main aspect, the invention relates to an encoded beaded or granulated polymer matrix for solid phase synthesis comprising beads or granules each comprising a plurality of spatially immobilised particles or vacuoles, wherein each particle or vacuole comprises at least one fluorescent dye compound of the invention as described herein, wherein each particle or vacuole is individually detectable.

Table 1 illustrates examples of preferred substituents comprising reactive groups. In some embodiments, two or three identical or different substituents comprising reactive groups can be attached via the amino groups. When the substituents comprising reactive groups are different and have different reactivities they permit stepwise reactions with e.g. polymer beads and/or biomolecules.

TABLE 1

| Amine used in | | |
|---|---|---|
| the substitu- | Converted to | |
| tion reaction | Reactive linker | Comments |
| 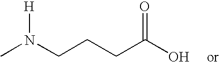 | N-succinimidyl ester Acid chloride Pentafluorophenol ester (or STP ester) | |
| 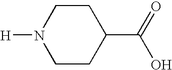 | | |
| 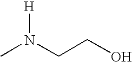 | Tosylate, mesylate, Halide | |
| 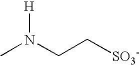 | Sulfonyl chloride | |
| — | Isothiocyanate | Amine |
| | | reactive |
| — | Maleimide | Thiol |
| | | reactive |

The ability to modify the solubility of the compounds according to the invention is important as poorly dissolvable compounds tend to self-aggregate or form noncovalent complexes with biomolecules. This may result in quenching of the fluorescence. Also, poorly dissolvable dyes may lead to precipitation/aggregation of the beads/biomolecules.

It is possible to modify the solubility of the compounds according to the invention by selecting preferred substituents, including the amino or substituted amine used in the reaction with the preferred substituents, including substituents comprising reactive groups.

In Table 2 some examples of amino groups with different polarity are illustrated.

TABLE 2

| Amine used in | |
|---|---|
| the substitu- | |
| tion reaction | |
| 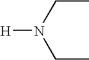 | Non polar dye (for use in non polar media) |
| 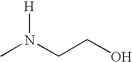 | Medium polar (could be use to synthesize “PEG” substituted derivatives) |
| 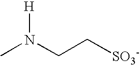 | Two of these anionic taurine substituents would give a (probably water soluble) dye with a total charge of minus one. |

TABLE 3

| ex. no. | X1-11 | Y | Z | R1 | R2 | R3 | R4 | R5 | R6 | R7 | R8 | R9 |
|---|---|---|---|---|---|---|---|---|---|---|---|---|
| 1 | H | NR₅R₆ | O | React-a | CH₃ | CH₃ | CH₃ | CH₃ | CH₃ | — | — | — |
| 2 | H | NR₅R₆ | O | Pol | CH₃ | CH₃ | CH₃ | CH₃ | CH₃ | — | — | — |
| 3 | H | NR₅R₆ | O | React-b | CH₃ | CH₃ | CH₃ | CH₃ | CH₃ | — | — | — |
| 4 | H | NR₅R₆ | O | React-a | CH₃ | React-b | CH₃ | CH₃ | CH₃ | — | — | — |
| 5 | H | NR₅R₆ | O | React-a | CH₃ | React-c | CH₃ | CH₃ | CH₃ | — | — | — |
| 6 | H | NR₅R₆ | O | React-a | CH₃ | React-b | CH₃ | React-c | CH₃ | — | — | — |
| 7 | Cl | NR₅R₆ | O | React-a | CH₃ | CH₃ | CH₃ | CH₃ | CH₃ | — | — | — |
| 8 | F | NR₅R₆ | O | React-a | CH₃ | CH₃ | CH₃ | CH₃ | CH₃ | — | — | — |
| 9 | H | OR₇ | O | CH₃ | CH₃ | CH₃ | CH₃ | — | — | React-a | — | — |
| 10 | H | SR₈ | O | CH₃ | CH₃ | CH₃ | CH₃ | — | — | — | React-a | — |
| 11 | H | R₉ | O | CH₃ | CH₃ | CH₃ | CH₃ | — | — | — | — | React-a |
| 12 | H | NR₅R₆ | O | React-a | C₂H₅ | C₂H₅ | C₂H₅ | C₂H₅ | C₂H₅ | — | — | — |
| 13 | H | NR₅R₆ | O | React-a | CH₃ | C₂H₄OH | CH₃ | C₂H₄OH | CH₃ | — | — | — |
| 14 | H | NR₅R₆ | O | React-a | CH₃ | —(CH₂)₃— | | —(CH₂)₃— | | — | — | — |
| 15 | H | NR₅R₆ | O | React-a | CH₃ | C₂H₄SO₃⁻ | CH₃ | C₂H₄SO₃⁻ | CH₃ | — | — | — |
| 16 | H | OR₇ | O | C₂H₄SO₃⁻ | CH₃ | C₂H₄SO₃⁻ | CH₃ | — | | React-a | — | — |
| 17 | H | OR₇ | O | C₂H₄SO₃⁻ | CH₃ | —(CH₂)₃— | | — | | React-a | — | — |
| 18 | H | NR₅R₆ | NR₁₀ | CH₃ | CH₃ | CH₃ | CH₃ | CH₃ | CH₃ | — | — | — |
| 19 | H | NR₅R₆ | NR₁₀ | CH₃ | CH₃ | CH₃ | CH₃ | CH₃ | CH₃ | — | — | — |
| 20 | H | NR₅R₆ | NR₁₀ | C₂H₅ | C₂H₅ | C₂H₅ | C₂H₅ | C₂H₅ | C₂H₅ | — | — | — |
| 21 | H | NR₅R₆ | NR₁₀ | C₂H₄OH | CH₃ | C₂H₄OH | CH₃ | C₂H₄OH | CH₃ | — | — | — |
| 22 | H | NR₅R₆ | NR₁₀ | C₂H₄SO₃⁻ | CH₃ | C₂H₄SO₃⁻ | CH₃ | —(CH₂)₃— | | — | — | — |
| 23 | H | NR₅R₆ | NR₁₀ | C₂H₄SO₃⁻ | CH₃ | —(CH₂)₃— | | —(CH₂)₃— | | — | — | — |
| 24 | H | NR₅R₆ | NR₁₀ | —(CH₂)₃— | | —(CH₂)₃— | | —(CH₂)₃— | | — | — | — |
| 25 | H | NR₅R₆ | NR₁₀ | CH₃ | CH₃ | CH₃ | CH₃ | CH₃ | CH₃ | — | — | — |
| 26 | H | NR₅R₆ | NR₁₀ | C₂H₅ | C₂H₅ | C₂H₅ | C₂H₅ | C₂H₅ | C₂H₅ | — | — | — |
| 27 | H | NR₅R₆ | NR₁₀ | C₂H₄OH | CH₃ | C₂H₄OH | CH₃ | C₂H₄OH | CH₃ | — | — | — |
| 28 | H | NR₅R₆ | NR₁₀ | —(CH₂)₃— | | —(CH₂)₃— | | —(CH₂)₃— | | — | — | — |
| 29 | H | NR₅R₆ | NR₁₀ | C₂H₄SO₃⁻ | CH₃ | —(CH₂)₃— | | —(CH₂)₃— | | — | — | — |
| 30 | H | F | NR₁₀ | CH₃ | CH₃ | CH₃ | CH₃ | — | — | — | — | — |

Abreviations:
Pol Polymer matrix preferably in the form of beads and based on polyethyleneglycols with amine or hydroxy termination
React-a —(CH₂)₃—COOH, preferably activated as N-succinimidyl ester, acid chloride or flurophenyl ester.
React-b —C₂H₄OH, preferably activated as tosylate or mesylate
React-c —(CH2)3-NH2, where the primary amine is preferably protected as a phtalimido or azido group

Apart from the preferred polymer matrices comprising PEGs which are mentioned under “Pol” in Table 3, “Pol” can further denote any of the polymers listed under the definition of this term.

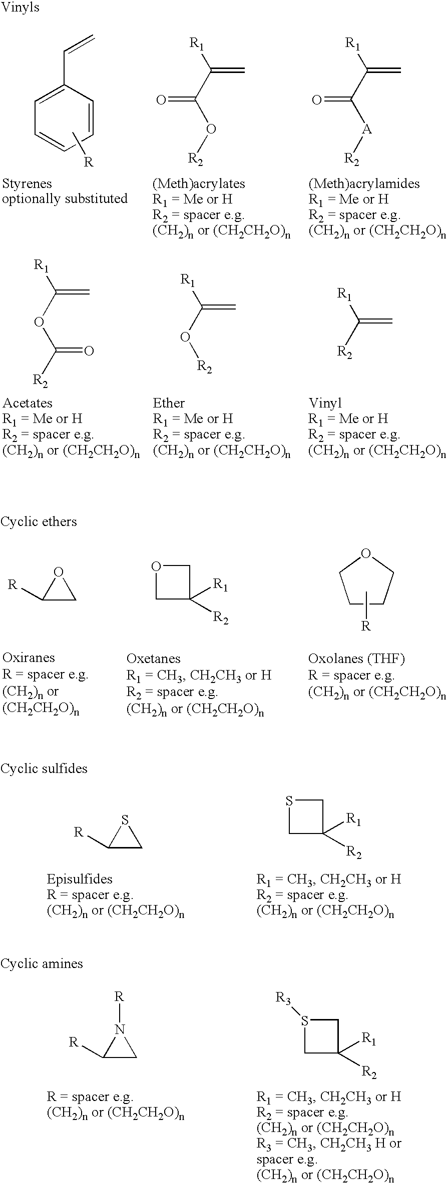

R, R₁, R₂, and R₃ denote the groups onto which the fluorescent dye compound can be attached. The numbering does not refer to fluorescent dye substitutents.

Synthesis of Triangulenium Compounds According to the Invention

One synthesis scheme for preparation for triangulenium derivatives according to the invention take advantage of the selective, facile, and stepwise substitution of the para methoxy groups in the readily available tris(2,4,6-trimethoxyphenyl)-carbenium ion (1, scheme 1). By this method, precursor compounds 4 in which R₁-R₆ are the same or different can be obtained in few and simple synthetic steps as outlined in Scheme 1 (Laursen, ibid.). The triangulenium compound 5 can be obtained by heating the corresponding compound 4 with LiI in NMP.

The synthesis of compounds according to the present invention can be performed based on the strategy disclosed herein above utilizing the reactivity of the tris(2,4,6-trimethoxyphenyl)-carbenium ion (1) towards amine nucleophiles.

Due to the stepwise nature of the substitution reaction, it is possible to obtain asymmetrical substituted triangulenium derivatives in a “one-pot” procedure as outlined in scheme 2.

Ring closure of compound 4a is performed by heating the compound in NMP with LiCl and butane acid.

This procedure is superior to the previously published procedure using NMP/LiI which results in methylation of the acid group.

### EXAMPLES

The invention is further described herein below in the non-limiting examples.

### Example 1

The synthesis of the compound designated as ATOTA-1 is outline in Scheme 2.

1 (BF₄⁻) is synthesized as described in the literature (Laursen et al. JACS 1998, 120, 12255-12263)

4a (BF₄⁻): 4-(methylamino)butyric acid hydrochloride (1.36 g; 8.8 mmol), 1 (5.0 g; 8.3 mmol), and diisopropylethylamine (5 mL) is dissolved in acetonitrile (120 mL). The reaction mixture is stirred at 30-35° C. in a dye nitrogen atmosphere for 22 h. Aqueous dimethylamine (40 mL of a 40% solution) is added and the reaction mixture is stirred for four more days. Solvent and excess dimethyl amine is removed in vacuo and the reminiscent material dissolved in chloroform. The chloroform solution was washed twice with brine and dried over MgSO₄ before evaporation of the solvent and reprecipitation from CH₂Cl₂/ether. Yield: 4.4 g (74%) dark powder.

MS (FAB+): m/z 624 (M+)

¹H-NMR (400 MHz, d6-DMSO): δ 8.34 (1H, s), 6.03 (2H, s), 5.83 (4H, s), 3.49 (2H, m), 3.46 (6H, s), 3.44 (12H, s), 3.12 (3H, s), 3.08 (12H, s), 1.94 (2H, t), 1.70 (2H, m).

5a (PF₆⁻): 4a (BF₄⁻) (2.3 g; 3.19 mmol), LiCl (2.4 g), and propanic acid (4.5 mL) was dissolved in NMP (150 mL) and heated to 180° C. for 80 minutes. Ethylacetate (ca 400 mL) was added in small portions to the cooled (25° C.) reaction mixture while stirring. The precipitate is filtered off and washed with ether, giving an orange solid (2.0 g). 1.8 g of this solid was dissolved in DMSO (ca 100 mL) and poured into 0.2 M aqueous KPF₆ solution (acidified with HCl, pH ca 2). The precipitate was filtered off and washed with pure water and recrystallized from ethanol/water (300 mL+120 mL), giving 1.40 g (69%) of 5a-PF₆⁻.

MS (FAB+): m/z 486 (M+) (at m/z 500 a signal from the methylester is observed, ca 10% in relative intensity)

¹H-NMR (400 MHz, d6-DMSO): δ 12.24 (1H, s), 6.40 (2H, s), 6.32 (2H, d, J=2 Hz), 6.29 (2H, d, J=2 Hz), 3.45 (2H, t, J=7 Hz), 3.09 (12H, s), 3.06 (3H, s), 2.37 (2H, t, J=7 Hz), 1.77 (2H, m). (at 3.66 ppm a singlet with a relative intensity of 0.2H is observed due to the methoxy group in the methylester).

ATOTA-1 (PF₆⁻): 5a (PF₆⁻) (0.80 g, 1.26 mmol) was dissolved in dry DMSO (50 mL) and diisopropylethylamine (0.30 g, 2.3 mmol) was added followed by TSTU (2-succinimido-1,1,3,3-tetramethyluronium tetrafluoroborate; 0.60 g, 2 mmol). The reaction mixture was stirred in a closed flask at room temperature for 2 hours, before it was poured into 0.2 M aqueous KPF₆ solution (250 mL) acidified with 2 M HCl solution (2 mL). The precipitate was filtered off and washed with pure water. The dried (vacuum over KOH) product was reprecipitated from acetonitrile (50 mL)/ether (200 mL). Yield: 0.81 g (90%) orange powder.

MS (FAB+): m/z 583 (M+) (at m/z 500 a signal from the methylester is observed, ca 10% in relative intensity)

¹H-NMR (400 MHz, d6-DMSO): δ 6.41 (2H, s), 6.30 (4H, s), 3.52 (2H, t, J=7 Hz), 3.09 (15H, m), 2.85 (6H, m), 2.50 (2H, m). (at 3.66 ppm a singlet with a relative intensity of 0.2H is observed due to the methoxy group in the methylester impurity)

The absorption and emission spectra of ATOTA-1 are given in FIG. 2. The dark line illustrates the measured absorption spectrum. The emission spectrum is illustrated by the gray line.

### Example 2

### Exposing Linked Fluorescent Dye to Harsh Conditions

General Methods

Reagents were obtained from Fluka and used without any purification. All solvents used were of HPLC grade kept over molecular sieves. ATOTA-1 was obtained from Bo Wegge Laursen, Nano-Science Center, Copenhagen University. The 5-28 μm beads were prepared by using a high-speed dispersion reactor. The beads (300-500 μm) were prepared in a 250 ml baffled glass reactor equipped with a dispersion stirrer. Washing and subsequent drying of beads was carried out by the following procedure: The beads were washed 10 times with ethanol, followed by 10 times wash with water, and finally another 10 times wash with ethanol and were then dried at room temperature under vacuum. In the following wash, washed, and washing refers to this procedure.

Thorough washing refers to a procedure comprising the steps of 10 times wash with di-chloromethane (DCM), 10 times wash with methanol (MeOH), 10 times wash with water, 10 times wash with di-methylformamide (DMF), another 10 times wash with MeOH, a further 10 times wash DCM, and finally drying at room temperature under vacuum

The fluorescence images were obtained with a Leica MZ12 stereo zoom microscope and a Leica digital camera with an exposure time of 2.3 sec and 0.8 times magnification. A mercury lamp equipped with a blue filter provided the broad banded excitation in the near UV range. A green filter was positioned between the sample and the lens of the microscope in order to filter away the incoming blue light. It was hereby obtained that only the fluorescence was recorded. The images of the fluorescent beads were recorded in DMF.

Synthesis of Partially Acryloylated (NH₂)₂PEG₅₀₀ and (NH₂)₂PEG₁₉₀₀

Acryloyl chloride (1.267 ml, 14 mmol) in DCM (12 ml) was added dropwise to a solution of (NH₂)₂PEG₅₀₀ (6.3 g, 10 mmol) in DCM (18 ml) at 0° C. with stirring. The reaction mixture was kept for 1 h at 20° C. The DCM was evaporated and drying in vacuum at 20° C. yielded the 70% acryloylated (NH₂)₂PEG₅₀₀ as colourless thick oil. The partially acryloylated (NH₂)₂PEG₁₉₀₀ was prepared by following the same procedure with (NH₂)₂PEG₁₉₀₀ (20 g, 10 mmol) in DCM (12 ml) and acryloyl chloride (1.267 ml, 14 mmol) in DCM (18 ml).

Preparation of (Acr)_{1.4}-(NH₂)₂PEG₁₉₀₀-Acrylamide Copolymer Beads.

The (Acr)_{1.4}(NH₂)₂PEG₁₉₀₀-acrylamide polymer beads were prepared by inverse suspension polymerisation. In order to prepare the beads having a size 300-500 μm, a 1.4 wt % of sorbitan monolaurate to the macromonomer was used as the suspension stabiliser. n-heptane was used as the suspension medium and was degassed with argon for 1 hour before the addition of monomers. In a typical synthesis procedure, a solution of (Acr)_{1.4}(NH₂)₂PEG₁₉₀₀ (7.3 g, 3.54 mmol) in water (21 ml) was degassed with argon for 30 min. Acrylamide (0.36 g, 5 mmol) in water (0.5 ml) was added to the degassed solution and the purging of argon was continued for 5 min. A solution of sorbitan monolaurate (0.1 ml) in DMF (1 ml) and the free radical initiator ammonium persulfate (300 mg) in water (2 ml) were added to the monomer mixture. The reaction mixture was then rapidly added to the suspension medium and stirred at 600 rpm at 70° C. After one min, TEMED (1.5 ml) was added to the reactor. The reaction was allowed to continue for 3 hours, the encoded beads formed were filtered through the sieves and the 300-500 μm fraction was collected. The beads were washed.

Labelling of Beads with ATOTA-1

Labelling of (Acr)_{1.4}(NH₂)₂PEG₁₉₀₀-Acrylamide (300-500 μm) beads with ATOTA-1

The beads (0.1 g, 0.2 mmol/g) were swollen in DMF (4 ml) for 1 h. The DMF was filtered off, the ATOTA-1 (0.0146 g, 0.02 mmol) in DMF (500 μL) was added to the swollen beads and the reaction mixture was kept at room temperature. After 24 h the beads were washed.

Stability of ATOTA-1 Labelled Beads in Acids, Bases, and Nucleophiles

The lyophilised ATOTA-1 labelled (Acr)_{1.4} (NH₂)₂PEG₁₉₀₀-Acrylamide (300-500 μm) beads were used for the stability studies. The beads (10 mg each, 0.2 mmol/g) were weighed into seven syringes fitted with a Teflon filter and treated with the reagents for 3 hours at room temperature.

Triflic anhydride: The beads were washed with 10 times with DCM, and then triflic anhydride (34 μl, 100 equiv) was added. After 3 h, the beads were filtered and thoroughly washed.

TMSOTf: The resin was washed with DCM (10×) and trimethylsilylmethyl trifluoromethanesulfonate (40 μl, 100 equiv) was added. After 3 h, the resin was filtered and thoroughly washed.

Trifluoroacetic acid: The dried resin was suspended in neat trifluoroacetic acid (100 μl) for 3 h. The resin was thoroughly washed.

Potassium hydroxide (KOH): The resin was washed with water (10×) and saturated aqueous KOH (200 μl) was added. After 3 hours, the resin was thoroughly washed.

Butyl lithium (BuLi): The resin was washed with DCM (10×) and THF (10×). Butyl lithium in heptane (54 μl, 10 equiv) was added. After 3 h, the resin was filtered and washed 10 times with tetra-hydrofourane and then washed thoroughly.

Boron-tri-fluoride (BF₃) in ethylether (Et₂O): The resin was washed with DCM (10×) and BF₃Et₂O (26 μl, 100 equiv) was added. After 3 h, the resin was filtered and thoroughly washed.

Piperidine: The resin was washed 10 times with DCM and 10 times with DMF. 20% piperidine in DMF (200 μl) was added. After 3 hours the resin was filtered and washed with THF and then washed thoroughly. 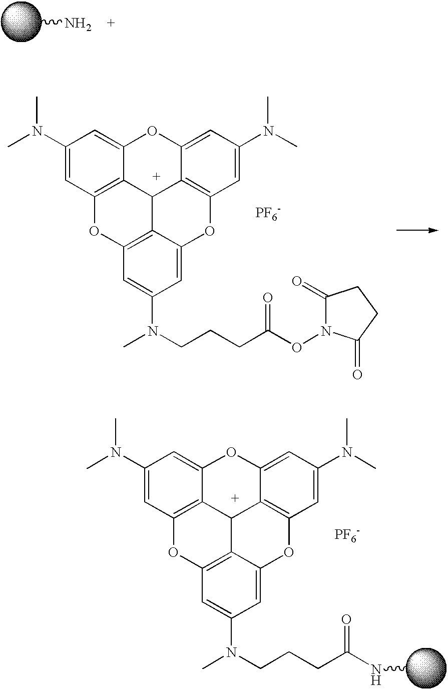
Results

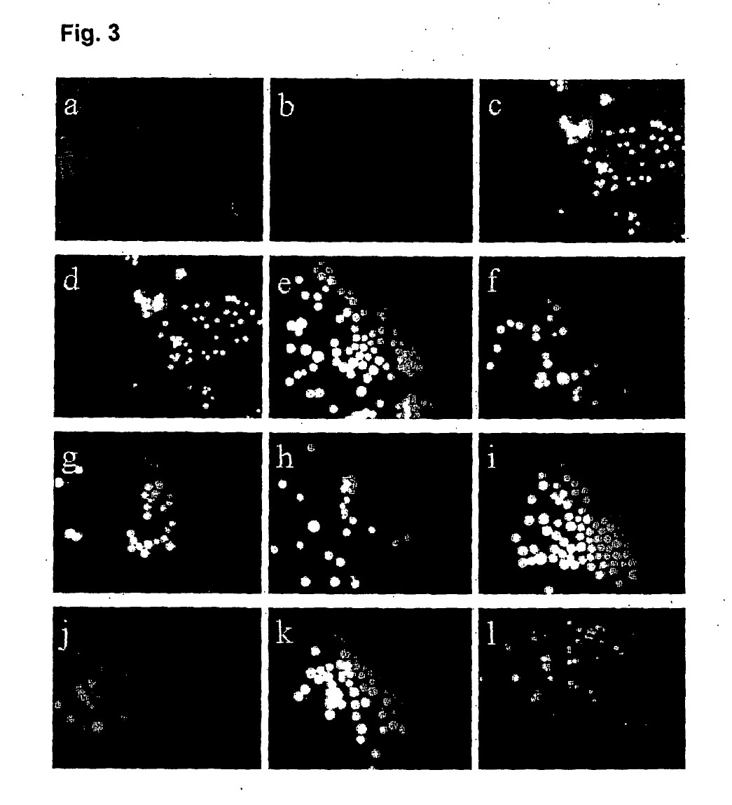

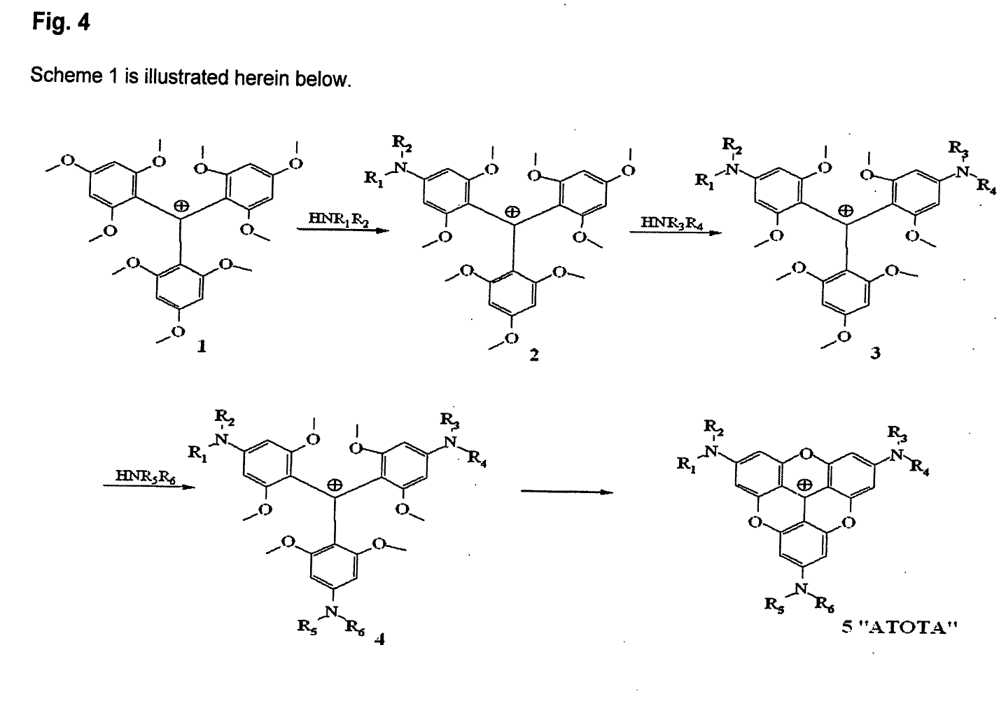

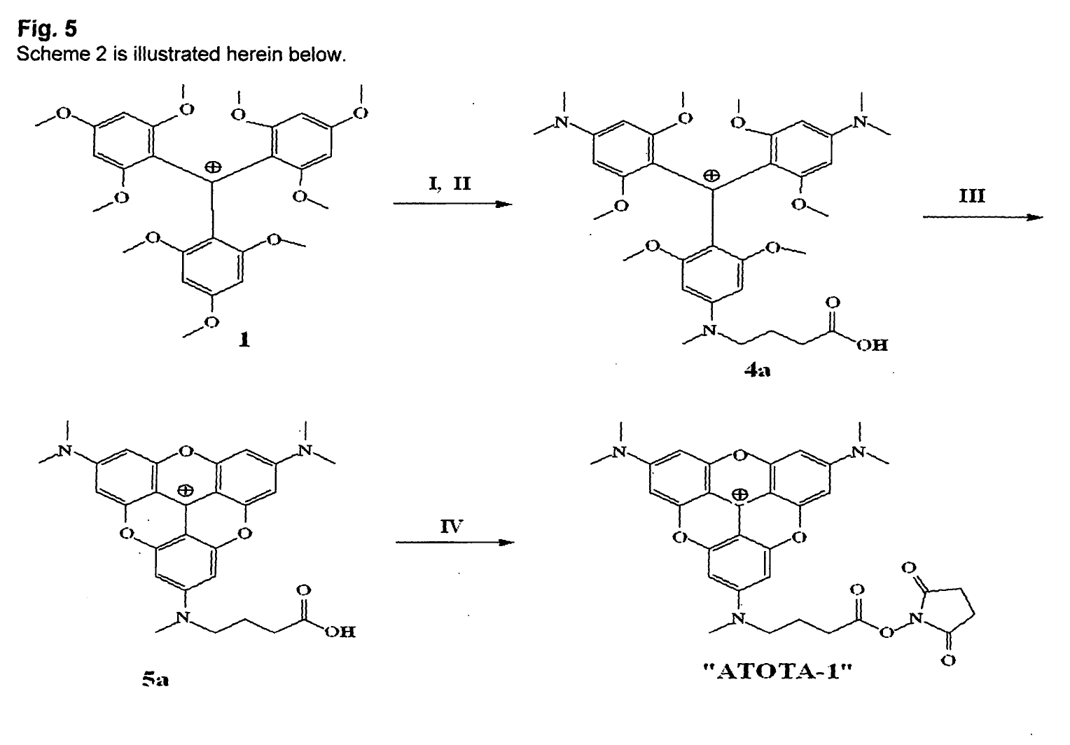

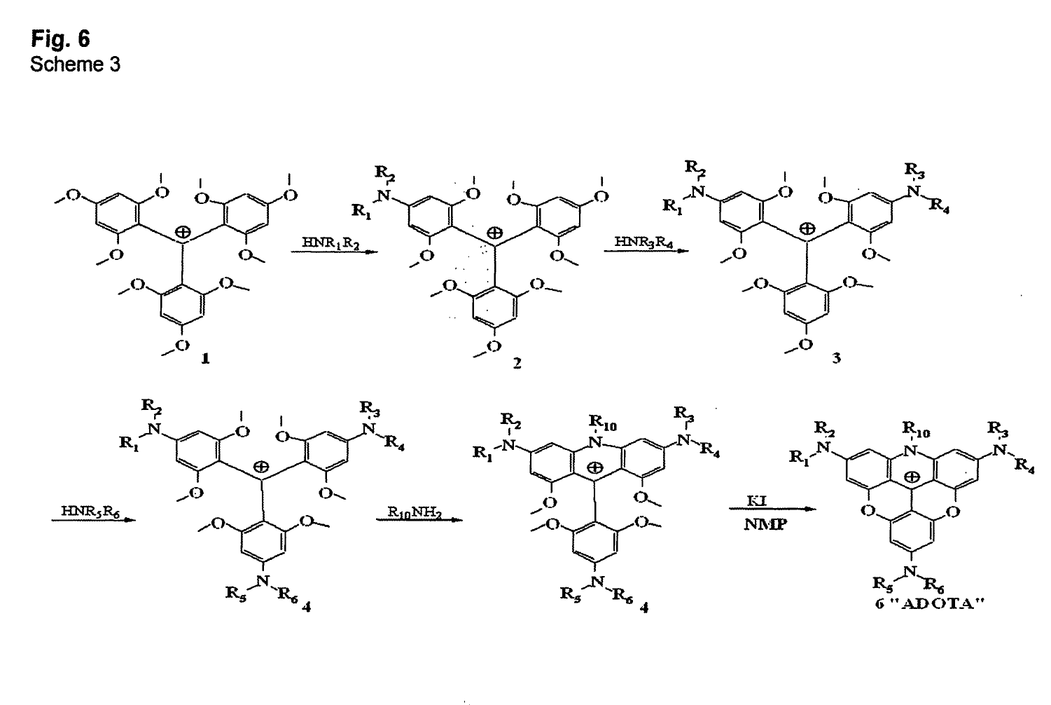

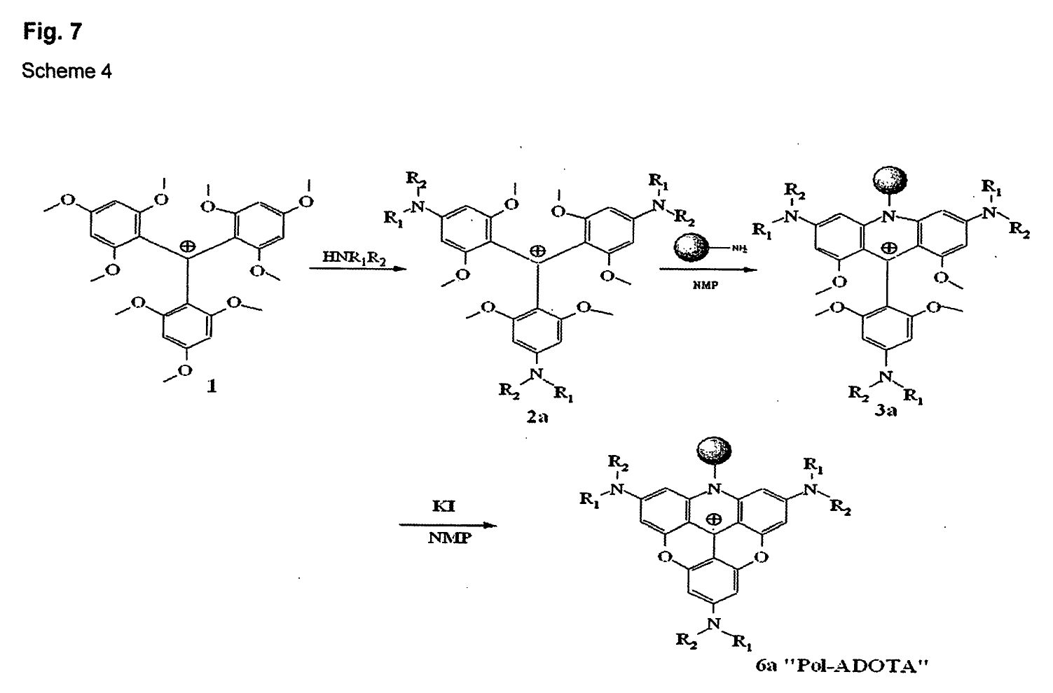

The ATOTA-1 labelled beads were suspended in DMF and analysed by fluorescence microscopy before and after exposure to the harsh environments specified above. The resulting fluorescence images are given in FIG. 3. It appears from the figure that the beads as well as the fluorescent dye were chemically stable in all reagents. Only the exposure to triflic anhydride seems to have reduced the fluorescence signal as the image of the beads after the triflic anhydride treatment shows a decreased intensity of fluorescence (FIG. 3j). In order to investigate whether this effect is reversible, the triflic anhydride treated beads were pH-neutralised by 20% DIPEA in DMF and subsequently analysed with the microscope set-up (FIG. 3l). The resulting image shows that the beads regain their fluorescence after neutralisation.

FIG. 3 illustrates images of beads suspended in DMF. a) unstained beads imaged without fluorescence filter, b) unstained beads imaged with green fluorescence filter, c) stained beads imaged without fluorescence filter, d) stained beads imaged with green fluorescence filter, e) stained beads exposed to BF₃ imaged with green fluorescence filter, f) stained beads exposed to BuLi imaged with green fluorescence filter, g) stained beads exposed to KOH imaged with green fluorescence filter, h) stained beads exposed to piperidine imaged with green fluorescence filter, i) stained beads exposed to tri-fluoroacetic acid imaged with green fluorescence filter, j) stained beads exposed to triflic anhydride imaged with green fluorescence filter, k) stained beads exposed to tri-methyl-silylmethyltriflate imaged with green fluorescence filter, l) stained beads exposed to triflic anhydride after neutralization imaged with green fluorescence filter.

### Example 3

Synthesis of labeled beads (6a) by in situ cyclisation onto amine polymer beads according to scheme 4.

0.2 g dry (Acr)_{1.4}-(NH₂)₂PEG₁₉₀₀-Acrylamide copolymer beads prepared according to the above described procedures were suspended in 5 ml of dry N-methylpyrolidone. 7.8 mg of hexamethoxy-tris(dimethylamine)triangulenium PF₆ (2a, R₁₋₆=methyl) was dissolved into the mixture, which was heated over night at 100 degrees C. 50 mg of anhydrous KI was added and the heating was continued for 8 days, followed by a 2 hr heating to 180 degrees C. and cooling to RT. The beads were filtered from the solution, washed 3 times with water, 3 times with acetone and resuspended in water. The resulting brownish yellow beads (6a, R₁₋₆=methyl) exhibited a strong green fluorescence when exited with blue light at 473 nm from a diode laser.

### LIST OF FIGURES

FIG. 1. Generic structure of the fluorescent dye compounds
FIG. 2. Absorption and emission spectra of ATOTA-1
FIG. 3. images of beads suspended in DMF.
Scheme 1. Synthesis scheme for preparation of triangulenium derivatives.
Scheme 2. “One-pot” synthesis scheme for preparation of asymmetrical substituted triangulenium derivatives.
Scheme 3. General synthesis of ADOTA (6) via stepwise substitution and cyclisation.
Scheme 4. Synthesis of polymer linked ADOTA by in situ substitution and cyclisation onto amine terminated polymer beads.

1. A fluorescent dye compound comprising the structure 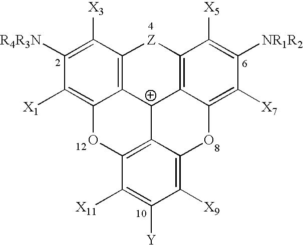
wherein X₁, X₃, X₅, X₇, X₉, and X₁₁ are independently H, Cl or F;
wherein Y is selected from the group consisting of H, Cl, F, NR₅R₆, OR₇, SR₈, and R₉;
wherein Z is O or NR₁₀, and
wherein R₁, R₂, R₃, R₄, R₅, R₆, R₇, R₈, R₉, and R₁₀ is independently H, an optionally substituted alkyl, an optionally substituted aryl, or an optionally polymerized substituted or unsubstituted monomer selected from the group consisting of styrene, acrylate, (meth)acrylate, hydroxymethyl(meth)acrylate, acrylamide, acetate, vinylacetate, vinylether, vinylpyrrolidone, oxirane, oxetane, oxolane, episulfide, thiotane, cyclic amine, or R₁ and R₂ together with the nitrogen to which they are attached form a substituted or unsubstituted heterocyclic, or R₃ and R₄ together with the nitrogen to which they are attached form a substituted or unsubstituted heterocyclic, or R₅ and R₆ together with the nitrogen to which they are attached form a substituted or unsubstituted heterocyclic, or wherein at least one of R₁, R₂, R₃, R₄, R₅, R₆, R₇, R₈, R₉, and R₁₀ comprises at least one reactive group or at least one reactive moiety, wherein the at least one reactive group is selected from the group consisting of vinyl, allyl, hydroxy, primary amine, secondary amine, carboxy, carbonyl, nitro, cyano, isothiocyanate, halogen, phosphonyl, sulphonate, sulphonyl, sulfamyl, and thiolyl, including any combination thereof, wherein the at least one reactive moiety is selected from the group consisting of an activated ester, such as N-succinimidyl ester, maleimide ester, or fluorophenol ester, such as pentafluorophenol ester; acid halide, such as acid chloride or acid bromide; sulfonyl halide, such as sulfonyl chloride or sulfonyl bromide; tosylate, mesylate, phtalimido, azido, an optionally polymerized substituted or unsubstituted styrene, an optionally polymerized substituted or unsubstituted acrylate, an optionally polymerized substituted or unsubstituted (meth)acrylate, an optionally polymerized substituted or unsubstituted hydroxymethyl(meth)acrylate; an optionally polymerized substituted or unsubstituted acrylamide, an optionally polymerized substituted or unsubstituted acetate, an optionally polymerized substituted or unsubstituted vinylacetate; an optionally polymerized substituted or unsubstituted vinylether; an optionally polymerized substituted or unsubstituted vinylpyrrolidone, an optionally polymerized substituted or unsubstituted oxirane; an optionally polymerized substituted or unsubstituted oxetane, an optionally polymerized substituted or unsubstituted oxolane; an optionally polymerized substituted or unsubstituted episulfide; an optionally polymerized substituted or unsubstituted thiotane; and an optionally polymerized substituted or unsubstituted cyclic amine, with the proviso that R₁ to R₆ are not all identical linear alkyls when Y is NR₅R₆ and Z is O, and that R₁ and R₂ are not both ethyl when NR₃R₄ and NR₅R₆ both constitute a morpholinyl ring.

2. The fluorescent dye compound according to claim 1, wherein X₁, X₃, X₅, X₇, X₉, and X₁₁ are all H.

3. The fluorescent dye compound according to claim 1, wherein X₁, X₃, X₅, X₇, X₉, and X₁₁ are all Cl.

4. The fluorescent dye compound according to claim 1, wherein X₁, X₃, X₅, X₇, X₉, and X₁₁ are all F.

5. The fluorescent dye compound according to claim 1, wherein Y is selected from H, Cl, and F.

6-8. (canceled)

9. The fluorescent dye compound according to claim 1, wherein Z is O or NR₁₀.

10-11. (canceled)

12. The fluorescent dye compound according to claim 1, wherein Y is selected from the group consisting of NR₅R₆, OR₇, SR₈, and R₉.

13-69. (canceled)

70. A fluorescent dye compound comprising the structure 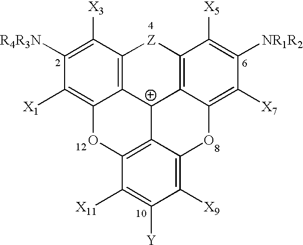
wherein X₁, X₃, X₅, X₇, X₉, and X₁₁ are independently H, Cl or F;
wherein Y is selected from the group consisting of H, Cl, F, NR₅R₆, OR₇, SR₈, and R₉;
wherein Z is O or NR₁₀, and
wherein substituents R₁ to R₁₀ are preferably selected independently from the group consisting of:
hydrido,
substituted and unsubstituted alkyl, substituted and unsubstituted haloalkyl, substituted and unsubstituted hydroxyalkyl, substituted and unsubstituted alkylsulfonyl, substituted and unsubstituted alkenyl,
halo,
substituted and unsubstituted alkoxy, substituted and unsubstituted alkoxyalkyl, substituted and unsubstituted haloalkoxy, substituted and unsubstituted haloalkoxyalkyl, substituted and unsubstituted aryl,
substituted and unsubstituted heterocyclic,
substituted and unsubstituted heteroaryl,
sulfonyl, substituted and unsubstituted alkylsulfonyl, substituted and unsubstituted arylsulfonyl, sulfamyl, sulfonamidyl, aminosulfonyl, substituted and unsubstituted N-alkylaminosulfonyl, substituted and unsubstituted N-arylaminosulfonyl, substituted and unsubstituted N,N-dialkylaminosulfonyl, substituted and unsubstituted N-alkyl-N-arylaminosulfonyl, substituted and unsubstituted N-alkylaminosulfonyl, substituted and unsubstituted N,N-dialkylaminosulfonyl, substituted and unsubstituted N-arylaminosulfonyl, substituted and unsubstituted N-alkyl-N-arylaminosulfonyl,
carboxy, substituted and unsubstituted carboxyalkyl, carbonyl, substituted and unsubstituted alkylcarbonyl, substituted and unsubstituted alkylcarbonylalkyl, substituted and unsubstituted alkoxycarbonyl, substituted and unsubstituted alkoxycarbonylalkyl,
aminocarbonyl, substituted and unsubstituted aminocarbonylalkyl, substituted and unsubstituted N-alkylaminocarbonyl, substituted and unsubstituted N-arylaminocarbonyl, substituted and unsubstituted N,N-dialkylaminocarbonyl, substituted and unsubstituted N-alkyl-N-arylaminocarbonyl, substituted and unsubstituted N-alkyl-N-hydroxyaminocarbonyl, substituted and unsubstituted N-alkyl-N-hydroxyaminocarbonylalkyl, substituted and unsubstituted N-alkylaminocarbonyl, substituted and unsubstituted N,N-dialkylaminocarbonyl, substituted and unsubstituted N-arylaminocarbonyl, substituted and unsubstituted N-alkyl-N-arylaminocarbonyl, substituted and unsubstituted aminocarbonylalkyl, substituted and unsubstituted N-cycloalkylaminocarbonyl,
substituted and unsubstituted aminoalkyl, substituted and unsubstituted alkylaminoalkyl,
amidino,
cyanoamidino,
substituted and unsubstituted heterocyclicalkyl,
substituted and unsubstituted aralkyl,
substituted and unsubstituted cycloalkyl,
substituted and unsubstituted cycloalkenyl,
substituted and unsubstituted alkylthio,
substituted and unsubstituted alkylsulfinyl,
substituted and unsubstituted N-alkylamino, substituted and unsubstituted N,N-dialkylamino,
substituted and unsubstituted arylamino, substituted and unsubstituted aralkylamino, substituted and unsubstituted N-alkyl-N-arylamino, substituted and unsubstituted N-aralkyl-N-alkylamino, substituted and unsubstituted N-arylaminoalkyl, substituted and unsubstituted N-aralkylaminoalkyl, substituted and unsubstituted N-alkyl-N-arylaminoalkyl, substituted and unsubstituted N-aralkyl-N-alkylaminoalkyl,
acyl, acylamino,
substituted and unsubstituted arylthio, substituted and unsubstituted aralkylthio,
substituted and unsubstituted aryloxy, substituted and unsubstituted aralkoxy,
substituted and unsubstituted haloaralkyl,
substituted and unsubstituted carboxyhaloalkyl,
substituted and unsubstituted alkoxycarbonylhaloalkyl,
substituted and unsubstituted aminocarbonylhaloalkyl,
substituted and unsubstituted alkylaminocarbonylhaloalkyl,
substituted and unsubstituted alkoxycarbonylcyanoalkenyl,
substituted and unsubstituted carboxyalkylaminocarbonyl,
substituted and unsubstituted aralkoxycarbonylalkylaminocarbonyl,
substituted and unsubstituted cycloalkylalkyl, and
substituted and unsubstituted aralkenyl,
wherein at least one of said substituents R1 to R10 comprises
a) one or more reactive groups selected from the group consisting of vinyl, allyl, hydroxy, primary amine, secondary amine, carboxy, carbonyl, nitro, cyano, isothiocyanate, halogen, phosphonyl, sulphonate, sulphonyl, sulfamyl, and thiolyl, or
b) one or more reactive moieties selected from the group consisting of an activated ester, such as N-succinimidyl ester, maleimide ester, or fluorophenol ester, including pentafluorophenol ester; acid halide, such as acid chloride or acid bromide; sulfonyl halide, such as sulfonyl chloride or sulfonyl bromide; tosylate, mesylate, phtalimido, azido, an optionally polymerized substituted or unsubstituted styrene, an optionally polymerized substituted or unsubstituted acrylate, an optionally polymerized substituted or unsubstituted (meth)acrylate, an optionally polymerized substituted or unsubstituted hydroxymethyl(meth)acrylate; an optionally polymerized substituted or unsubstituted acrylamide, an optionally polymerized substituted or unsubstituted acetate, an optionally polymerized substituted or unsubstituted vinylacetate; an optionally polymerized substituted or unsubstituted vinylether; an optionally polymerized substituted or unsubstituted vinylpyrrolidone, an optionally polymerized substituted or unsubstituted oxirane; an optionally polymerized substituted or unsubstituted oxetane, an optionally polymerized substituted or unsubstituted oxolane; an optionally polymerized substituted or unsubstituted episulfide; an optionally polymerized substituted or unsubstituted thiotane; and an optionally polymerized substituted or unsubstituted cyclic amine,
with the proviso that R₁ to R₆ are not all identical linear alkyls when Y is NR₅R₆ and Z is O, and that R₁ and R₂ are not both ethyl when NR₃R₄ and NR₅R₆ both constitute a morpholinyl ring.

71. The fluorescent dye compound according to claim 70, wherein X₁, X₃, X₅, X₇, X₉, and X₁₁ are all H.

72. The fluorescent dye compound according to claim 70, wherein X₁, X₃, X₅, X₇, X₉, and X₁₁ are all Cl.

73. The fluorescent dye compound according to claim 70, wherein X₁, X₃, X₅, X₇, X₉, and X₁₁ are all F.

74. The fluorescent dye compound according to claim 70, wherein Y is selected from H, Cl, and F.

75-77. (canceled)

78. The fluorescent dye compound according to claim 70, wherein Y is selected from the group consisting of NR₅R₆, OR₇, SR₈, and R₉.

79-82. (canceled)

83. The fluorescent dye compound according to claim 78, wherein Y comprises a reactive group capable of undergoing polymerization.

84. (canceled)

85. The fluorescent dye compound according to claim 78, wherein Y is a linker capable of linking the dye compound to a polymer matrix.

86. (canceled)

87. The fluorescent dye compound according to claim 70, wherein Z is O or NR₁₀.

88-90. (canceled)

91. Method for producing a polymer matrix comprising a fluorescent dye, said method comprising the steps of providing a monomer or a polymer matrix and reacting the fluorescent dye compound according to claim 1, or a precursor thereof, with the monomer or polymer matrix, and optionally reacting the fluorescent dye compound precursor to obtain the fluorescent dye compound, and further optionally polymerizing the monomers to obtain a polymer matrix.

92-93. (canceled)

94. An encoded beaded or granulated polymer matrix for solid phase synthesis comprising beads or granules each comprising a plurality of spatially immobilised particles or vacuoles, wherein each particle or vacuole comprises at least one fluorescent dye compound according to claim 1, wherein each particle or vacuole is individually detectable.

95. Method for producing a polymer matrix comprising a fluorescent dye, said method comprising the steps of providing a monomer or a polymer matrix and reacting the fluorescent dye compound according to claim 70, or a precursor thereof, with the monomer or polymer matrix, and optionally reacting the fluorescent dye compound precursor to obtain the fluorescent dye compound, and further optionally polymerizing the monomers to obtain a polymer matrix.

96. An encoded beaded or granulated polymer matrix for solid phase synthesis comprising beads or granules each comprising a plurality of spatially immobilised particles or vacuoles, wherein each particle or vacuole comprises at least one fluorescent dye compound according to claim 70, wherein each particle or vacuole is individually detectable.
